(19) United States
(12) Patent Application Publication
(10) Pub. No.: US 2026/0357290 A1
(43) Pub. Date: Nov. 12, 2026

(54) AAV EVOLUTION AT SINGLE-CELL RESOLUTION USING SPLIT-SEQ

(71) Applicant: THE CHILDREN'S HOSPITAL OF PHILADELPHIA, Philadelphia, PA (US)

(72) Inventors: Beverly DAVIDSON, Philadelphia, PA (US); Paul RANUM, Philadelphia, PA (US); Yonghong CHEN, Philadelphia, PA (US); Ashley ROBBINS, Philadelphia, PA (US)

(73) Assignee: THE CHILDREN'S HOSPITAL OF PHILADELPHIA, Philadelphia, PA (US)

(21) Appl. No.: 19/112,453

(22) PCT Filed: Sep. 19, 2023
(86) PCT No.: PCT/US2023/074565

§ 371 Date: Mar. 17, 2025

Related U.S. Application Data
(60) Provisional application No. 63/407,826, filed on Sep. 19, 2022.

(51) Int. Cl. C12N 15/86; C07K 14/005; C12N 15/10

(52) U.S. Cl. CPC C12N 15/86; C07K 14/005; C12N 15/1065; C12N 15/1096; C12N 2750/14122; C12N 2750/14143

(57) ABSTRACT

Provided herein are compositions and methods that allow for the identification of specific cell types that are transduced by a barcoded adeno-associated virus (AAV). These compositions and methods can be used to identify the cellular tropism of AAVs having modified capsid proteins that comprise targeting peptides.

### REFERENCE TO RELATED APPLICATIONS

The present application claims the priority benefit of U.S. provisional application No. 63/407,826, filed Sep. 19, 2022, the entire contents of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Sep. 19, 2023, is named CHOPP0057WO_ST26.xml and is 28.221 bytes in size.

### BACKGROUND

### 1. Field

The present invention relates generally to the fields of molecular biology, virology, and medicine. More particularly, it concerns compositions and methods for determining the cellular tropism of AAV capsid proteins having targeting peptides.

### 2. Description of Related Art

One of the primary challenges in detecting the transduction of barcoded AAV capsids at the single cell level is detecting both the mRNA sequences that provide information about cell identity and simultaneously detecting delivered AAV capsid DNA or expressed RNA. Methods are needed that allow for the identified of the specific cell type transduced by a given barcoded AAV capsid.

### SUMMARY

Provided herein are barcoded RNA expression constructs that, when packaged into an AAV and delivered to cells, express mRNA sequences with an identifying barcode that is distinct from the modified region of capsid DNA sequence. In one embodiment, a recombinant adeno-associated virus (rAAV) vector is provided that comprises an expression cassette encoding a barcode sequence that is operably linked to an RNA polymerase III promoter. In one embodiment, a population of recombinant adeno-associated virus (rAAV) vectors, each rAAV vector comprising an expression cassette encoding a barcode sequence that is operably linked to an RNA polymerase III promoter. The population of vectors may comprise a 1:1, 1:5, 1:10, 1:50, 1:100, 1:500, or at least 1:1000 ratio of barcode sequences to vectors. In one embodiment, provided herein are populations of recombinant adeno-associated virus (rAAV) vectors, wherein each rAAV vector independently comprises (i) a modified adeno-associated virus (AAV) Cap gene encoding a modified AAV capsid protein comprising a targeting peptide and (ii) an expression cassette encoding a barcode sequence that is operably linked to an RNA polymerase III promoter, wherein each targeting peptide and each barcode is uniquely paired.

The barcode sequence may be at least 9, at least 12, at least 15, at least 18, or at least 21 nucleotides long. The barcode sequence may be 9-21 nucleotides long, 12-21 nucleotides long, 15-21 nucleotides long, 9-18 nucleotides long, 9-15 nucleotides long, or 9-12 nucleotides long. The barcode sequence may be 9, 10, 11, 12, 13, 14, 15, 16, 17, 18, 19, 20, or 21 nucleotides long. The barcode sequence may be flanked by sequences capable of hybridizing to and activating a padlock probe. The barcode sequences each, independently, comprise a (NNNT)ₙ sequence.

The RNA polymerase III promoter may be a type III RNA polymerase III promoter. The RNA polymerase promoter may be a U6 snRNA gene promoter, H1 RNA gene promoter, or 7SK gene promoter.

The rAAV vector(s) may further comprise a reverse transcription primer binding site positioned 3′ of the barcode sequence and an enrichment primer binding site positioned 5′ of the barcode sequence. The expression cassette may comprise a sequence that is identical to, at least 90% identical to, or at least 95% identical to SEQ ID NO: 7.

The rAAV vector(s) may further comprise a modified adeno-associated virus (AAV) Cap gene encoding a modified AAV capsid protein comprising a targeting peptide. The modified AAV capsid protein may be a modified AAV1 capsid protein, a modified AAV2 capsid protein, or a modified AAV9 capsid protein. The targeting peptide may be three to ten amino acids in length. The targeting peptide may be 3, 4, 5, 6, 7, 8, 9, or 10 amino acids in length.

If the modified AAV capsid protein is derived from an AAV1 capsid protein (see SEQ ID NO: 1), then the targeting peptide may be inserted after residue 590 of the AAV1 capsid protein. The targeting peptide may be flanked by linker sequences, wherein the linker sequences on each side of the targeting peptides are two or three amino acids long. The linker sequences may be SSA on the N-terminal side of the targeting peptide and AS on the C-terminal side of the targeting peptide. The modified AAV1 capsid protein may have a sequence identical to, at least 90% identical to, or at least 95% identical to SEQ ID NO: 4.

If the modified AAV capsid protein is derived from an AAV2 capsid protein (see SEQ ID NO: 2), then the targeting peptide may be inserted after residue 587 of the AAV2 capsid protein. The targeting peptide may be flanked by linker sequences, wherein the linker sequences on each side of the targeting peptides are two or three amino acids long. The linker sequences may be AAA on the N-terminal side of the targeting peptide and AA on the C-terminal side of the targeting peptide. The modified AAV2 capsid protein may have a sequence identical to, at least 90% identical to, or at least 95% identical to SEQ ID NO: 5.

If the modified AAV capsid protein is derived from an AAV9 capsid protein (see SEQ ID NO: 3), then the targeting peptide may be inserted after residue 588 of the AAV9 capsid protein. The targeting peptide may be flanked by linker sequences, wherein the linker sequences on each side of the targeting peptides are two or three amino acids long. The linker sequences may be AAA on the N-terminal side of the targeting peptide and AS on the C-terminal side of the targeting peptide. The modified AAV9 capsid protein may have a sequence identical to, at least 90% identical to, or at least 95% identical to SEQ ID NO: 6.

A population of rAAV vectors may be provided, where the population comprises a plurality of capsid protein targeting peptides, wherein each capsid protein targeting peptide is paired with more than one barcode sequence. A population of rAAV vectors may be provided, where the population comprises a plurality of capsid protein targeting peptides, wherein all rAAVs having the same barcode sequence also have the same capsid protein targeting peptide. In other words, multiple RNAbc sequences represent a single AAV peptide insertion sequence. This feature enables the use a “randomer” sequence during plasmid generation, making the methods provided herein more high throughput because it is not necessary to individually clone each RNAbc-AAV peptide insertion combination.

Also provided herein are cells comprising the rAAV vectors of the present embodiments. The cells may be mammalian cells. The cells may be human cells. The cells may by in vitro or in vivo.

Also provided herein are library preparation techniques designed to simultaneously barcode and recover both mRNAs and AAV-derived RNAs. In one embodiment, provided herein is a method of determining the cellular tropism of a recombinant adeno-associated virus (rAAV) having a modified AAV capsid protein comprising a targeting peptide, the method comprising (i) contacting a variety of cell types with the modified rAAV vector of any one of the present embodiments; (ii) identifying cells transduced by the modified rAAV vector based on the presence of the barcode sequence; and (iii) detecting the expressed transcriptome of each transduced cell, on a cell-by-cell basis, thereby determining the cellular tropism of the modified rAAV. In one embodiment, provided herein is a method of determining the cellular tropism of a recombinant adeno-associated virus (rAAV) having a modified AAV capsid protein comprising a targeting peptide, the method comprising (i) contacting a variety of cell types with a population of rAAV vectors provided herein; (ii) detecting both the expressed transcriptome and the rAAV that transduced each cell, on a cell-by-cell basis, and (iii) determining which cell types were transduced by which modified rAAV vector, thereby determining the cellular tropism of the modified rAAV.

The contacting in (i) may be performed in vitro or in vivo.

Detecting the expressed transcriptome and the rAAV in (ii) may comprise: (a) isolating, fixing and permeabilizing the nuclei of the cells contacted in (i); (b) dividing the nuclei into a plurality of first aliquots; (c) reverse transcribing the expressed cellular RNA molecules within the nuclei using primers comprising a poly(T) sequence to form complementary DNA (cDNA) molecules, and reverse transcribing the expressed rAAV RNA molecules within the nuclei using primers comprising a sequence sufficient to hybridize to and reverse transcribe the barcode sequence within the expression cassette to form AAV amplicons; (d) labeling the cDNA molecules and AAV amplicons with a first 5′ barcode, wherein the first 5′ barcode for the primers in each first aliquot is unique such that the cDNA molecules and AAV amplicons from the nuclei of each aliquot can be identified in comparison to the cDNA molecules and AAV amplicons from the nuclei of all other aliquots; (e) combining the plurality of first aliquots; (f) dividing the combined plurality of first aliquots into a plurality of second aliquots; (g) ligating a second 5′ barcode to the 5′ ends of the cDNA molecules and the AAV amplicons for form dual barcoded cDNA molecules and AAV amplicons, wherein the second 5′ barcode in each second aliquot is unique; (h) combining the plurality of second aliquots; (i) dividing the combined plurality of first aliquots into a plurality of third aliquots; (j) ligating a third 5′ barcode to the 5′ ends of the cDNA molecules and the AAV amplicons to form triple barcoded cDNA molecules and AAV amplicons, wherein the third 5′ barcode in each third aliquot is unique; (k) combining the plurality of third aliquots; (l) lysing the nuclei to release the cDNA molecules and the AAV amplicons from within the nuclei to form a lysate; and (m) sequencing the cDNA molecules and the AAV amplicons to thereby detect both the expressed transcriptome and the rAAV that transduced each cell.

The cDNA molecules and AAV amplicons may be labeled with the first 5′ barcode simultaneously with the reverse transcription, wherein the reverse transcription primers comprising the first 5′ barcode. The nuclei may be fixed and permeabilized at below about 8 °C at below about 7 °C, at below about 6 °C, at below about 5 °C, at below about 4 °C, at below about 3 °C at below about 2 °C, or at below about 1 °C. The majority of the triple barcoded cDNA molecules and AAV molecules from a single nucleus may comprise the same series of barcodes. The majority of the triple barcoded cDNA molecules and AAV molecules from a single nucleus may have a unique series of barcodes as compared to the triple barcoded cDNA molecules and AAV molecules from other nuclei. The cell types may be determined based on the expressed transcriptome.

Sequencing the cDNA molecules and the AAV amplicons comprises preparing a sequencing library, where preparing the sequencing library may comprise: (i) adding a common adapter sequence to the 3′-ends of the cDNA molecules and AAV amplicons; (ii) performing full-length cDNA and AAV amplicon amplification; (iii) fragmenting the amplified full-length cDNA and AAV amplicons; (iv) end repairing and A-tailing the fragmented cDNA and AAV amplicons; (v) ligating an adaptor to the 5′ ends of the end repaired and A-tailed cDNA and AAV amplicons; and (vi) performing a sample index PCR to add sequencing adapters and dual indices to the adaptor ligated cDNA and AAV amplicons.

Sequencing the AAV amplicons comprises preparing a sequencing library enriched for the AAV amplicons, where the preparing the sequencing library may comprise: (i) adding a common adapter sequence to the 3′-ends of the cDNA molecules and AAV amplicons; (ii) performing full-length amplification of cDNA and AAV amplicons; (iii) performing an AAV amplicon enrichment amplification with a forward primer that hybridizes to the AAV amplicons upstream of the AAV barcode, wherein the forward primer has a 5′ phosphate; (iv) A-tailing the AAV amplicons having a 5′ phosphate; (v) ligating an adaptor to the 5′ ends of the A-tailed AAV amplicons; and (vi) performing a sample index PCR to add sequencing adapters and dual indices to the adaptor ligated AAV amplicons.

The common adapter sequence may be added to the 3′-end of the cDNA molecules and AAV amplicons by template switching.

The sequencing may be paired-end sequencing, amplicon sequencing, single-cell RNA sequencing, or in situ sequencing.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F. Design of a double barcode containing AAV cargo. (A) Schematic depicting each element in barcoded expression construct (SEQ ID NO: 7) component of the AAV cargo. (B) Cartoon schematic depicting the layout of the construct shown in FIG. 1A within the packaged AAV genome, and relative to the AAV Cap gene sequence that has been modified to contain a peptide insertion. (C) A gel image showing amplification of the RNAbc sequence after reverse transcription using printers pr749 and 750. (D) A schematic highlighting the two distinct DNA barcodes present, the RNAbc and the peptide insertion into the Cap sequence. (E and F) Sanger sequencing spanning each insertion confirms successful creation of this dual barcoded construct. In FIG. 1E, the five sequences, from top to bottom, are SEQ ID NOs: 15-19, respectively. In FIG. 1F, the top sequence is SEQ ID NO: 20 and the bottom four sequences are all SEQ ID NO: 21.

FIGS. 2A-2B. Adaption of Split-Pool Ligation-based whole-Transcriptome Sequencing (SPLiT-seq) for AAV.RNAbc detection. (A) Schematic showing the procedural steps of single-nuclei combinatorial barcoding. (B) Schematic showing the procedural steps for next-generation library preparation for whole-transcriptome and AAV.RNAbc amplicon sequencing.

FIGS. 3A-3D. Single-cell RNA-Seq results showing detection of the RNA barcode (RNAbc) expressed after transfection of HEK 293 cells. (A) UMAP unbiased clustering of SPLiT-Seq barcoded single-cells from an experiment where HEK 293 cells were transfected with plasmids containing either AAV.RNAbc or AAV noBarcode. (B) Unique UMI counts obtained from Illumina sequencing reads after amplification and library preparation. (C) UMAP unbiased clustering of SPLiT-Seq barcoded single-cells showing only cells that received the AAV.RNAbc treatment. Heatmap shading indicates the log UMI counts originating from the AAV.RNAbc amplicon. (D) UMAP unbiased clustering of SPLiT-Seq barcoded single-cells showing only cells that received the AAV.eGFP treatment. Heatmap shading indicates the log UMI counts originating from the AAV.eGFP amplicon.

FIGS. 4A-4D. In vivo application of SPLiT-Seq. (A) Schematic showing procedural steps. (B) Seurat cell annotation containing cDNA expression and AAV transduction information. (C) Identification of transduced single cells. (D) Assessment of AAV transduction status within a single cell type of interest.

FIGS. 5A-5C. Tools for analysis of transduction performance. (A) Transduction performance by tissue. (B) Transduction performance by cell type (C) Transduction performance spatially visualized using UMAP unbiased clustering.

### DETAILED DESCRIPTION

Provided herein are barcoded RNA expression constructs that, when packaged into an AAV and delivered to cells, express mRNA sequences with an identifying barcode that is distinct from the modified region of capsid DNA sequence. Also provided herein are library preparation techniques designed to simultaneously barcode and recover both mRNAs and AAV-derived RNAs. Finally, custom software pipelines are provided, which integrate the publicly available SPLiT-Seq demultiplexing pipeline with a pipeline for counting AAV amplicon sequences.

### I. ADENO-ASSOCIATED VIRUS (AAV) VECTORS

Adeno-associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive- or negative-sensed. The approximately 4.7 kb genome of AAV consists of one segment of single stranded DNA of either plus or minus polarity. The ends of the genome are short inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for plasmid propagation and production, but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter that comprises an AAV capsid. The AAV particle comprises an icosahedral symmetry comprised of three related capsid proteins, VP1, VP2 and VP3, which interact together to form the capsid. The genome of most native AAVs often contain two open reading frames (ORFs), sometimes referred to as a left ORF and a right ORF. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles. In certain embodiments, the genome of an AAV particle encodes one, two or all three VP1, VP2 and VP3 polypeptides.

The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short inverted terminal repeats (ITR) which have the potential to fold into T-shaped hairpin structures that serve as the origin of viral DNA replication. Accordingly, the genome of an AAV comprises one or more (e.g. a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The term “recombinant,” as a modifier of vector, such as recombinant viral, e.g., lenti- or parvo-virus (e.g., AAV) vectors, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV, retroviral, or lentiviral vector would be where a nucleic acid sequence that is not normally present in the wild-type viral genome is inserted within the viral genome. An example of a recombinant nucleic acid sequence would be where a nucleic acid (e.g., gene) encodes an inhibitory RNA cloned into a vector, with or without 5′, 3′ and/or intron regions that the gene is normally associated within the viral genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild type genome of a virus by using molecular methods to remove part of the wild type genome from the virus, and replacing with a non-native nucleic acid, such as a nucleic acid sequence. Typically, for example, for AAV, one or both inverted terminal repeat (ITR) sequences of the AAV genome are retained in the recombinant AAV vector. A “recombinant” viral vector (e.g., rAAV) is distinguished from a viral (e.g., AAV) genome, since part of the viral genome has been replaced with a non-native sequence with respect to the viral genomic nucleic acid such a nucleic acid encoding a transactivator or nucleic acid encoding an inhibitory RNA or nucleic acid encoding a therapeutic protein. Incorporation of such non-native nucleic acid sequences therefore defines the viral vector as a “recombinant” vector, which in the case of AAV can be referred to as a “rAAV vector.”

In certain embodiments, an AAV (e.g., a rAAV) comprises two ITRs. In certain embodiments, an AAV (e.g., a rAAV) comprises a pair of ITRs. In certain embodiments, an AAV (e.g., a rAAV) comprises a pair of ITRs that flank (i e, are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is a rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 and AAV-2i8.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV11, AAV12, Rh10, Rh74 or AAV-2i8 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-2i8 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use, administration or delivery of an rAAV1, rAAV2, rAAV3, rAAV4, rAAV5, rAAV6, rAAV7, rAAV8, rAAV9, rAAV10, rAAV11, rAAV12, rRh10, rRh74 or rAAV-2i8 particle.

In certain embodiments, a method herein comprises use, administration or delivery of a rAAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV9 particle comprises an AAV9 capsid. In certain embodiments a rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments a rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, a rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In some embodiments, the rAAV comprise a modified capsid, wherein the modified capsid comprises a targeting peptide. In certain embodiments, the AAV is AAV1, AAV2, or AAV9. An exemplary wildtype reference AAV1 capsid protein sequence is provided in SEQ ID NO: 1. An exemplary wildtype reference AAV2 capsid protein sequence is provided in SEQ ID NO: 2. An exemplary wildtype reference AAV9 capsid protein sequence is provided in SEQ ID NO: 3. In certain aspects, the targeting peptide is inserted at position 59X) of the AAV1 capsid, position 587 of the AAV2 capsid, or position 588 of the AAV9 capsid. An exemplary modified AAV1 capsid protein sequence is provided in SEQ ID NO: 4, which shows the targeting peptide insertion after position 590 as SSAX₇AS, where the leading SSA and the trailing AS are linker sequences and X₇ represents the targeting peptide. An exemplary modified AAV2 capsid protein sequence is provided in SEQ ID NO: 5, which shows the targeting peptide insertion after position 587 as AAAX₇AA, where the leading AAA and the trailing AA are linker sequences and X₇ represents the targeting peptide. An exemplary modified AAV9 capsid protein sequence is provided in SEQ ID NO: 6, which shows the targeting peptide insertion after position 588 as AAAX₇AS, where the leading AAA and the trailing AS are linker sequences and X₇ represents the targeting peptide.

TABLE 1
AAV capsid sequences

| AAV | | SEQ ID |
|---|---|---|
| Capsid | Sequence | NO: |
| AAV1 | MAADGYLPDWLEDNLSEGIREWWDLKPGAPKPKANQQKQDDGRGLVLPGYKY | 1 |
| Capsid | LGPFNGLDKGEPVNAADAAALEHDKAYDQQLKAGDNPYLRYNHADAEFQERL | |
| | QEDTSFGGNLGRAVFQAKKRVLEPLGLVEEGAKTAPGKKRPVEQSPQEPDSS | |
| | SGIGKTGQQPAKKRINFGQTGDSESVPDPQPIGEPPATPAAVGPTTMASGGG | |
| | APMADNNEGADGVGNASGNWHCDSTWIGDRVITTSTRTWALPTYNNHLYKQI | |
| | SSASTGASNDNHYFGYSTPWGYFDFNRFHCHFSPRDWQRLINNNWGFRPKRL | |
| | NFKLENIQVKEVTINDGVTTIANNLTSTVQVESDSEYQLPYVLGSAHQGCLP | |
| | PFPADVEMIPQYGYLTLNNGSQAVGRSSFYCLEYFPSQMLRTGNNFTFSYTF | |
| | EEVPFHSSYAHSQSLDRLMNPLIDQYLYYLNRTQNQSGSAQNKDLLFSRGSP | |
| | AGMSVQPKNWLPGPCYRQQRVSKTKTDNNNSNETWTGASKYNINGRESIINP | |
| | GTAMASHKDDEDKFFPMSGVMIFGKESAGASNTALDNVMITDEEEIKATNPV | |
| | ATERFGTVAVNFQSSSTDPATGDVHAMGALPGMVWQDRDVYLQGPIWAKIPH | |
| | TDGHFHPSPLMGGFGLKNPPPQILIKNTPVPANPPAEFSATKFASFITQYST | |
| | GQVSVEIEWELQKENSKRWNPEVQYTSNYAKSANVDFTVDNNGLYTEPRPIG | |
| | TRYLTRPL | |
| AAV2 | MAADGYLPDWLEDTLSEGIRQWWKLKPGPPPPKPAERHKDDSRGLVLPGYKY | 2 |
| Capsid | LGPFNGLDKGEPVNEADAAALEHDKAYDRQLDSGDNPYLKYNHADAEFQERL | |
| | KEDTSFGGNLGRAVFQAKKRVLEPLGLVEEPVKTAPGKKRPVEHSPVEPDSS | |
| | SGTGKAGQQPARKRLNFGQTGDADSVPDPQPLGQPPAAPSGLGTNTMATGSG | |
| | APMADNNEGADGVGNSSGNWHCDSTWMGDRVITTSTRTWALPTYNNHLYKQI | |
| | SSQSGASNDNHYEGYSTPWGYEDFNRFHCHFSPRDWQRLINNNWGFRPKRLN | |
| | FKLFNIQVKEVTQNDGITTIANNLTSTVQVETDSEYQLPYVLGSAHQGCLPP | |
| | FPADVFMVPQYGYLTLNNGSQAVGRSSFYCLEYFPSQMLRTGNNFTFSYTFE | |
| | DVPFHSSYAHSQSLDRLMNPLIDQYLYYLSRINTPSGTTTQSRLQFSQAGAS | |
| | DIRDQSRNWLPGPCYRQQRVSKTSADNNNSEYSWTGATKYHLNGRDSLVNPG | |
| | PAMASHKDDEEKFFPQSGVLIFGKQGSEKTNVDIEKVMITDEEEIRTINPVA | |
| | TEQYGSVSTNLQRGNRQAATADVNTQGVLPGMVWQDRDVYLQGPIWAKIPHT | |
| | DGHFHPSPLMGGFGLKHPPPQILIKNTPVPANPSTTFSAAKFASFITQYSTG | |
| | QVSVEIEWELQKENSKRWNPEIQYTSNYNKSVNVDFTVDINGVYSEPRPIGT | |
| | RYLTRNL | |
| AAV9 | MAADGYLPDWLEDNLSEGIREWWALKPGAPQPKANQQHQDNARGLVLPGYKY | 3 |
| Capsid | LGPGNGLDKGEPVNAADAAALEHDKAYDQQLKAGDNPYLKYNHADAEFQERL | |
| | KEDTSFGGNLGRAVEQAKKRLLEPLGLVEEAAKTAPGKKRPVEQSPQEPDSS | |
| | AGIGKSGAQPAKKRLNFGQTGDTESVPDPQPIGEPPAAPSGVGSLTMASGGG | |
| | APVADNNEGADGVGSSSGNWHCDSQWLGDRVITTSTRTWALPTYNNHLYKQI | |
| | SNSTSGGSSNDNAYFGYSTPWGYFDFNRFHCHFSPRDWQRLINNNWGFRPKR | |
| | LNFKLENIQVKEVIDNNGVKTIANNLTSTVQVFTDSDYQLPYVLGSAHEGCE | |
| | PPFPADVEMIPQYGYLTINDGSQAVGRSSFYCLEYFPSQMLRTGNNFQFSYE | |
| | FENVPFHSSYAHSQSLDRLMNPLIDQYLYYLSKTINGSGQNQQTLKFSVAGP | |
| | SNMAVQGRNYIPGPSYRQQRVSTTVTQNNNSEFAWPGASSWALNGRNSLMNP | |
| | GPAMASHKEGEDRFFPLSGSLIFGKQGTGRDNVDADKVMITNEEEIKTTNPV | |
| | ATESYGQVATNHQSAQAQAQTGWVQNQGILPGMVWQDRDVYLQGPIWAKIPH | |
| | TDGNFHPSPIMGGFGMKHPPPQILIKNTPVPADPPTAFNKDKLNSFITQYST | |
| | GQVSVEIEWELQKENSKRWNPEIQYTSNYYKSNNVEFAVNTEGVYSEPRPIG | |
| | TRYLTRNL | |
| AAV1 | MAADGYLPDWLEDNLSEGIREWWDLKPGAPKPKANQQKQDDGRGLVLPGYKY | 4 |
| Capsid | LGPFNGLDKGEPVNAADAAALEHDKAYDQQLKAGDNPYLRYNHADAEFQERL | |
| with | QEDTSFGGNLGRAVFQAKKRVLEPLGLVEEGAKTAPGKKRPVEQSPQEPDSS | |
| targeting | SGIGKTGQQPAKKRINFGQTGDSESVPDPQPLGEPPATPAAVGPTTMASGGG | |
| peptide | APMADNNEGADGVGNASGNWHCDSTWLGDRVITTSTRTWALPTYNNHLYKQI | |
| | SSASTGASNDNHYEGYSTPWGYFDFNRFHCHFSPRDWQRLINNNWGFRPKRL | |
| | NFKLFNIQVKEVTINDGVTTIANNLTSTVQVESDSEYQLPYVLGSAHQGCLP | |
| | PFPADVEMIPQYGYLTLNNGSQAVGRSSFYCLEYFPSQMLRTGNNFTESYTF | |
| | EEVPFHSSYAHSQSLDRLMNPLIDQYLYYLNRTQNQSGSAQNKDLLFSRGSP | |
| | AGMSVQPKNWLPGPCYRQQRVSKTKTDNNNSNETWTGASKYNLNGRESIINP | |
| | GTAMASHKDDEDKFFPMSGVMIFGKESAGASNTALDNVMITDEEEIKATNPV | |
| | ATERFGTVAVNFQSSSTDSSAXXXXXXXASPATGDVHAMGALPGMVWQDRDV | |
| | YLQGPIWAKIPHTDGHFHPSPLMGGFGLKNPPPQILIKNTPVPANPPAEFSA | |
| | TKFASFITQYSTGQVSVEIEWELQKENSKRWNPEVQYTSNYAKSANVDFTVD | |
| | NNGLYTEPRPIGTRYLTRPL | |
| AAV2 | MAADGYLPDWLEDTLSEGIRQWWKLKPGPPPPKPAERHKDDSRGLVLPGYKY | 5 |
| Capsid | LGPFNGLDKGEPVNEADAAALEHDKAYDRQLDSGDNPYLKYNHADAEFQERL | |
| with | KEDTSFGGNLGRAVFQAKKRVLEPLGLVEEPVKTAPGKKRPVERSPVEPDSS | |
| targeting | SGTGKAGQQPARKRLNFGQTGDADSVPDPQPLGQPPAAPSGLGTNTMATGSG | |
| peptide | APMADNNEGADGVGNSSGNWHCDSTWMGDRVITTSTRIWALPTYNNHLYKQI | |
| | SSQSGASNDNHYFGYSTPWGYFDFNRFHCHFSPRDWQRLINNNWGFRPKRLN | |
| | EKLENIQVKEVTQNDGITTIANNITSTVQVETDSEYQLPYVLGSAHQGCLPP | |
| | FPADVFMVPQYGYLTLNNGSQAVGRSSFYCLEYFPSQMLRTGNNFTFSYTFE | |
| | DVPFHSSYAHSQSLDRLMNPLIDQYLYYLSRTNTPSGTTTQSRLQFSQAGAS | |
| | DIRDQSRNWLPGPCYRQQRVSKTSADNNNSEYSWTGATKYHLNGRDSLVNPG | |
| | PAMASHKDDEEKFFPQSGVLIFGKQGSEKTNVDIEKVMITDEEEIRTTNPVA | |
| | TEQYGSVSTNLQRGNAAAXXXXXXXAARQAATADVNTQGVLPGMVWQDRDVY | |
| | LQGPIWAKIPHIDGHFHPSPLMGGFGLKHPPPQILIKNTPVPANPSTTFSAA | |
| | KFASFITQYSTGQVSVEIEWELQKENSKRWNPEIQYTSNYNKSVNVDFTVDT | |
| | NGVYSEPRPIGTRYLTRNL | |
| AAV9 | MAADGYLPDWLEDNISEGIREWWALKPGAPQPKANQQHQDNARGLVLPGYRY | 6 |
| Capsid | LGPGNGLDKGEPVNAADAAALEHDKAYDQQLKAGDNPYLKYNHADAEFQERL | |
| with | KEDTSFGGNLGRAVFQAKKRLLEPLGLVEEAAKTAPGKKRPVEQSPQEPDSS | |
| targeting | AGIGKSGAQPAKKRINFGQTGDTESVPDPQPIGEPPAAPSGVGSLTMASGGG | |
| peptide | APVADNNEGADGVGSSSGNWHCDSQWLGDRVITTSTRTWALPTYNNHLYKQI | |
| | SNSTSGGSSNDNAYFGYSTPWGYFDFNRFHCHFSPRDWQRLINNNWGFRPKR | |
| | LNEKLFNIQVKEVTDNNGVKTIANNLTSTVQVETDSDYQLPYVLGSAHEGCL | |
| | PPFPADVEMIPQYGYLTLNDGSQAVGRSSFYCLEYFPSQMLRTGNNFQFSYE | |
| | FENVPFHSSYAHSQSLDRLMNPLIDQYLYYLSKTINGSGQNQQTLKFSVAGP | |
| | SNMAVQGRNYIPGPSYRQQRVSTTVTQNNNSEFAWPGASSWALNGRNSLMNP | |
| | GPAMASHKEGEDRFFPLSGSLIFGKQGTGRDNVDADKVMITNEEEIKTTNPV | |
| | ATESYGQVATNHQSAQAAAXXXXXXXASAQAQTGWVQNQGILPGMVWQDRDV | |
| | YLQGPIWAKIPHTDGNFHPSPLMGGFGMKHPPPQILIKNTPVPADPPTAFNK | |
| | DKINSFITQYSTGQVSVEIEWELQKENSKRWNPEIQYTSNYYKSNNVEFAVN | |
| | TEGVYSEPRPIGTRYLTRNL | |

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11. AAV12, AAV-rh74. AAV-rh10 or AAV-2i8, as long as they retain one or more desired ITR functions (e.g. ability to form a hairpin, which allows DNA replication; integration of the AAV DNA into a host cell genome; and/or packaging, if desired).

In certain embodiments, a rAAV2 particle comprises one or two ITRs (e.g., a pair of ITRs) that are at least 75% or more identical. e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication, integration of the AAV DNA into a host cell genome; and/or packaging, if desired).

In certain embodiments, a rAAV9 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication, integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent, and requires additional protein machinery to self-replicate. Thus, a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, HEK293 (readily available through. e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral E1a and E1b genes is used to generate recombinant AAV particles. The modified HEK293 cell line is readily transfected, and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

### II. METHODS FOR DETERMINING MODIFIED AAV CELLULAR TROPISM

Methods of uniquely labeling or barcoding molecules within a nucleus or a plurality of nuclei are provided herein. It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

The term “binding” is used broadly throughout this disclosure to refer to any form of attaching or coupling two or more components, entities, or objects. For example, two or more components may be bound to each other via chemical bonds, covalent bonds, ionic bonds, hydrogen bonds, electrostatic forces, Watson-Crick hybridization, etc.

One aspect of the disclosure relates to methods of labeling nucleic acids. In some embodiments, the methods may comprise labeling nucleic acids in a first nucleus. The methods may comprise: (a) generating complementary DNAs (cDNAs) from cellular RNAs and/or AAV.RNAbc amplicons within a plurality of nuclei by reverse transcribing RNAs using a reverse transcription primer comprising a 5′ overhang sequence; (b) dividing the plurality of nuclei into a number (n) of aliquots, (c) providing a plurality of barcode tags to each of the n aliquots, wherein each labeling sequence of the plurality of barcode tags provided into a given aliquot is the same, and wherein a different labeling sequence is provided into each of the n aliquots; (d) binding at least one of the cDNAs and/or AAV.RNAbc amplicons in each of the n aliquots to the barcode tags; (e) combining the n aliquots; and (f) repeating steps (b), (c), (d), and (e) with the combined aliquot. In some aspects, two different reverse transcription primers are used, where the first of the reverse transcription primers comprises a poly(A) hybridizing sequence (i.e., a poly(T) sequence) and where the second of the reverse transcription primers comprises a sequence capable of hybridizing to the RNA expressed from the AAV barcoding expression construct (i.e., AAV.RNAbc transcripts) downstream of the barcode sequence.

In certain embodiments, each barcode tag may comprise a first strand including a 3′ hybridization sequence extending from a 3′ end of a labeling sequence and a 5′ hybridization sequence extending from a 5′ end of the labeling sequence. Each barcode tag may also comprise a second strand including an overhang sequence. The overhang sequence may include (i) a first portion complementary to at least one of the 5′ hybridization sequence and the 5′ overhang sequence and (ii) a second portion complementary to the 3′ hybridization sequence. In some embodiments, the barcode tag (e.g., the final nucleic acid tag) may comprise a capture agent such as, but not limited to, a 5′ biotin. A cDNA or AAV.RNAbc amplicon labeled with a 5′ biotin-comprising barcode tag may allow or permit the attachment or coupling of the cDNA or AAV.RNAbc amplicon to a streptavidin-coated magnetic bead. In some other embodiments, a plurality of beads may be coated with a capture strand (i.e., a nucleic acid sequence) that is configured to hybridize to a final sequence overhang of a barcode tag. In yet some other embodiments, cDNA or AAV.RNAbc amplicon molecules may be purified or isolated by use of a commercially available kit (e.g., an RNEASY™ kit).

In various embodiments, step (f) (i.e., steps (b), (c), (d), and (e)) may be repeated a number of times sufficient to generate a unique series of labeling sequences for the cDNAs and AAV.RNAbc amplicons in the first nucleus. Stated another way, step (f) may be repeated a number of times such that the cDNAs and AAV.RNAbc amplicons in the first nucleus may have a first unique series of labeling sequences, the cDNAs and AAV.RNAbc amplicons in a second nucleus may have a second unique series of labeling sequences, the cDNAs and AAV.RNAbc amplicons in a third nucleus may have a third unique series of labeling sequences, and so on. The methods of the present disclosure may provide for the labeling of cDNA and AAV.RNAbc amplicon sequences from single nuclei with unique barcodes, wherein the unique barcodes may identify or aid in identifying the cell from which the cDNA and AAV.RNAbc amplicon originated. In other words, a portion, a majority, or substantially all of the cDNA and AAV RNAbc amplicons from a single cell may have the same barcode, and that barcode may not be repeated in cDNA or AAV.RNAbc amplicons originating from one or more other cells in a sample (e.g., from a second cell, a third cell, a fourth cell, etc.).

In some embodiments, barcoded cDNA and AAV.RNAbc amplicons can be mixed together and sequenced (e.g., using NGS), such that data can be gathered regarding RNA expression and AAV transduction at the level of a single cell. For example, certain embodiments of the methods of the present disclosure may be useful in assessing, analyzing, or studying the cellular tropism of a modified AAV (i.e., particular cell type that any given modified AAV capsid selective or specifically targets.

As discussed above, an aliquot or group of nuclei can be separated into different reaction vessels or containers and a first set of barcode tags can be added to the plurality of cDNA transcripts and AAV.RNAbc transcripts. Vessels or containers can also be referred to herein as receptacles, samples, and wells. Accordingly, the terms vessel, container, receptacle, sample, and well may be used interchangeably herein. The aliquots of nuclei can then be regrouped, mixed, and separated again and a second set of barcode tags can be added to the first set of barcode tags. In various embodiments, the same barcode tag may be added to more than one aliquot of nuclei in a single or given round of labeling. However, after repeated rounds of separating, tagging, and repooling, the cDNAs and AAV.RNAbc amplicons of each nuclei may be bound to a unique combination or sequence of barcode tags that identify a single nucleus. In some embodiments, nuclei in a single sample may be separated into a number of different reaction vessels. For example, the number of reaction vessels may include four 1.5 ml microcentrifuge tubes, a plurality of wells of a 96-well plate, a plurality of wells of a 384-well plate, or another suitable number and type of reaction vessels.

In certain embodiments, step (f) (i.e., steps (b), (c), (d), and (e)) may be repeated a number of times wherein the number of times is selected from 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, etc. In certain other embodiments, step (f) may be repeated a sufficient number of times such that the cDNAs and AAV.RNAbc amplicons of each nucleus would be likely to be bound to a unique sequence of barcode tags. The number of times may be selected to provide a greater than 50% likelihood, greater than 90% likelihood, greater than 95% likelihood, greater than 99% likelihood, or some other probability that the cDNAs and AAV.RNAbc amplicons in each nucleus are bound to a unique sequence of barcode tags. In yet other embodiments, step (f) may be repeated some other suitable number of times.

In some embodiments, the methods of labeling nucleic acids in the first nucleus may comprise fixing the plurality of nuclei prior to step (a). For example, components of a nucleus may be fixed or cross-linked such that the components are immobilized or held in place. The plurality of nuclei may be fixed using formaldehyde in phosphate buffered saline (PBS). The plurality of nuclei may be fixed, for example, in about 1-4% formaldehyde in PBS. In various embodiments, the plurality of nuclei may be fixed using methanol (e.g., 100% methanol) at about −20 °C or at about 25 °C. In various other embodiments, the plurality of nuclei may be fixed using methanol (e.g., 100% methanol), at between about −20 °C and about 25 °C. In yet various other embodiments, the plurality of nuclei may be fixed using ethanol (e.g., about 70-100% ethanol) at about −20 °C or at room temperature. In yet various other embodiments, the plurality of nuclei may be fixed using ethanol (e.g., about 70-100% ethanol) at between about −20 °C and room temperature. In still various other embodiments, the plurality of nuclei may be fixed using acetic acid, for example, at about −20 °C. In still various other embodiments, the plurality of nuclei may be fixed using acetone, for example, at about −20 °C. Other suitable methods of fixing the plurality of nuclei are also within the scope of this disclosure.

In certain embodiments, the methods of labeling nucleic acids in the first nuclei may comprise permeabilizing the plurality of nuclei prior to step (a). For example, holes or openings may be formed in nuclear membranes of the plurality of nuclei. TRITON™ X-100 may be added to the plurality of nuclei, followed by the optional addition of HCl to form the one or more holes. About 0.2% TRITON™ X-100 may be added to the plurality of nuclei, for example, followed by the addition of about 0.1 N HCl. In certain other embodiments, the plurality of nuclei may be permeabilized using ethanol (e.g., about 70% ethanol), methanol (e.g., about 100% methanol), Tween 20 (e.g., about 0.2% Tween 20), and/or NP-40 (e.g., about 0.1% NP-40). In various embodiments, the methods of labeling nucleic acids in the first nucleus may comprise fixing and permeabilizing the plurality of nuclei prior to step (a).

In some embodiments, the methods of labeling nucleic acids in the first nucleus may comprise ligating at least two of the barcode tags that are bound to the cDNAs and/or AAV.RNAbc amplicons. Ligation may be conducted before or after the lysing and/or the nucleic acid purification steps. Ligation can comprise covalently linking the 5′ phosphate sequences on the barcode tags to the 3′ end of an adjacent strand or barcode tag such that individual tags are formed into a continuous, or substantially continuous, barcode sequence that is bound to the 3′ end of the cDNA sequence. In various embodiments, a double-stranded DNA or RNA ligase may be used with an additional linker strand that is configured to hold a barcode tag together with an adjacent nucleic acid in a “nicked” double-stranded conformation. The double-stranded DNA or RNA ligase can then be used to seal the “nick.” In various other embodiments, a single-stranded DNA or RNA ligase may be used without an additional linker. In certain embodiments, the ligation may be performed within the plurality of nuclei.

In certain other embodiments, the methods may comprise lysing the plurality of nuclei (i.e., breaking down the nuclear structure) to release the cDNAs and/or AAV.RNAbc amplicons from within the plurality of nuclei, for example, after step (f). In some embodiments, the plurality of nuclei may be lysed in a lysis solution (e.g., 10 mM Tris-HCl (pH 7.9), 50 mM EDTA (pH 7.9), 0.2 M NaCl, 2.2% SDS, 0.5 mg/ml ANTI-RNase (a protein ribonuclease inhibitor; AMBION®) and 1000 mg/ml proteinase K (AMBION®)), for example, at about 55 °C for about 1-3 hours with shaking (e.g., vigorous shaking). In some other embodiments, the plurality of nuclei may be lysed using ultrasonication and/or by being passed through an 18-25 gauge syringe needle at least once. In yet some other embodiments, the plurality of nuclei may be lysed by being heated to about 70-90 °C. For example, the plurality of nuclei may be lysed by being heated to about 70-90 °C for about one or more hours. The cDNAs and/or AAV.RNAbc amplicons may then be isolated from the lysed nuclei. In some embodiments, RNase H may be added to the cDNA and AAV.RNAbc amplicons to remove RNA. The methods may further comprise ligating at least two of the barcode tags that are bound to the released cDNAs and AAV.RNAbc amplicons. In some other embodiments, the methods of labeling nucleic acids in the first cell may comprise ligating at least 3, 4, 5, 6, 7, 8, 9, 10, 11, 12, 13, 14, 15, 16, 17, 18, 19, 20, 21, 22, 23, 24, 25, 30, 35, 40, 45, 50, etc. of the barcode tags that are bound to the cDNAs and AAV.RNAbc amplicons.

In various embodiments, the methods of labeling nucleic acids in the first nucleus may comprise removing one or more unbound barcode tags (e.g., washing the plurality of nuclei). For example, the methods may comprise removing a portion, a majority, or substantially all of the unbound barcode tags. Unbound barcode tags may be removed such that further rounds of the disclosed methods are not contaminated with one or more unbound barcode tags from a previous round of a given method. In some embodiments, unbound barcode tags may be removed via centrifugation. For example, the plurality of nuclei can be centrifuged such that a pellet of nuclei is formed at the bottom of a centrifuge tube. The supernatant (i.e., liquid containing the unbound barcode tags) can be removed from the centrifuged nuclei. The nuclei may then be resuspended in a buffer (e.g., a fresh buffer that is free or substantially free of unbound barcode tags). In another example, the plurality of nuclei may be coupled or linked to magnetic beads that are coated with an antibody that is configured to bind the nuclear membrane. The plurality of nuclei can then be pelleted using a magnet to draw them to one side of the reaction vessel.

As discussed above, the plurality of nuclei can be repooled and the method can be repeated any number of times, adding more barcode tags to the cDNAs and AAV.RNAbc amplicons creating a unique set of barcode tags that can serve to identify the cDNAs and AAV.RNAbc amplicons as originating from the same cell. As more and more rounds are added, the number of paths that a nucleus can take increases and consequently the number of possible unique barcodes tag sequences that can be created also increases. Given enough rounds and divisions, the number of possible barcodes will be much higher than the number of nuclei, resulting in each nucleus likely having a unique barcode. For example, if the division took place in a 96-well plate, after 4 divisions there would be 96⁴=84,934,656 possible barcodes.

In some embodiments, the cDNA reverse transcription primer may be configured to reverse transcribe all, or substantially all, RNA in a cell (e.g., a random hexamer with a 5′ overhang). In some other embodiments, the cDNA reverse transcription primer may be configured to reverse transcribe RNA having a poly(A) tail (e.g., a poly(dT) primer, such as a dT(15) primer, with a 5′ overhang). In yet some other embodiments, the cDNA reverse transcription primer may be configured to reverse transcribe predetermined RNAs (e.g., a transcript-specific primer). For example, the cDNA reverse transcription primer may be configured to barcode specific transcripts such that fewer transcripts may be profiled per cell, but such that each of the transcripts may be profiled over a greater number of cells.

In some embodiments, the AAV.RNAbc reverse transcription primer may be configured to reverse transcribe RNA expressed from the AAV barcoding expression construct (i.e., AAV.RNAbc transcripts). For example, the AAV.RNAbc reverse transcription primer may be configured to hybridize to the AAV.RNAbs transcript downstream of the barcode sequence.

Reverse transcription may be conducted or performed on the plurality of nuclei. In certain embodiments, reverse transcription may be conducted on a fixed and/or permeabilized plurality of nuclei. In some embodiments, variants of M-MuLV reverse transcriptase may be used in the reverse transcription. Any suitable method of reverse transcription is within the scope of this disclosure. For example, a reverse transcription mix may include a reverse transcription primer including a 5′ overhang and the reverse transcription primer may be configured to initiate reverse transcription and/or to act as a binding sequence for barcode tags. In some other embodiments, a portion of a reverse transcription primer that is configured to bind to RNA and/or initiate reverse transcription may comprise one or more of the following: a random hexamer, a septamer, an octomer, a nonamer, a decamer, a poly(T) stretch of nucleotides, and/or one or more gene specific primers.

Another aspect of the disclosure relates to methods of uniquely labeling RNA molecules within a plurality of nuclei. The methods may include: (a) fixing and permeabilizing a first plurality of nuclei prior to step (b), wherein the first plurality of nuclei may be fixed and permeabilized at below about 8 °C; (b) reverse transcribing the RNA molecules within the first plurality of cells to form complementary DNA (cDNA) molecules and AAV.RNAbc amplicons within the first plurality of nuclei, wherein reverse transcribing the RNA molecules includes coupling primers to the RNA molecules, wherein the primers include at least one of a poly(T) sequence or a sequence capable of hybridizing to the RNA expressed from the AAV barcoding expression construct (i.e., AAV.RNAbc transcripts) downstream of the barcode sequence; (c) dividing the first plurality of nuclei including cDNA molecules and AAV.RNAbc amplicons into at least two primary aliquots, the at least two primary aliquots including a first primary aliquot and a second primary aliquot; (d) providing primary barcode tags to the at least two primary aliquots, wherein the primary barcode tags provided to the first primary aliquot are different from the primary barcode tags provided to the second primary aliquot; (e) coupling the cDNA molecules and AAV.RNAbc amplicons within each of the at least two primary aliquots with the provided primary barcode tags; (f) combining the at least two primary aliquots; (g) dividing the combined primary aliquots into at least two secondary aliquots, the at least two secondary aliquots including a first secondary aliquot and a second secondary aliquot; (h) providing secondary barcode tags to the at least two secondary aliquots, wherein the secondary barcode tags provided to the first secondary aliquot are different from the secondary barcode tags provided to the second secondary aliquot; (i) coupling the cDNA molecules and AAV.RNAbc amplicons within each of the at least two secondary aliquots with the provided secondary barcode tags; (j) repeating steps (f), (g), (h), and (i) with subsequent aliquots, wherein the final barcode tags include a capture agent; (k) combining final aliquots; (l) lysing the first plurality of nuclei to release the cDNA molecules and AAV.RNAbc amplicons from within the first plurality of nuclei to form a lysate; and/or (m) adding a protease inhibitor and/or a binding agent to the lysate such that the cDNA molecules and AAV.RNAbc amplicons bind the binding agent.

The method may further include dividing the combined final aliquots into at least two final aliquots, the at least two final aliquots including a first final aliquot and a second final aliquot. In some embodiments, the first plurality of nuclei may be fixed and permeabilized at below about 8 °C below about 7 °C below about 6 °C below about 5 °C, at about 4 °C, below about 4 °C, below about 3 °C below about 2 °C below about 1 °C, or at another suitable temperature. In certain embodiments, the methods may include splitting the nuclei. For example, following the last or final round of barcoding (via ligation), the nuclei can be pooled before lysis and then the nuclei can be split into different lysate aliquots. Each lysate aliquot may include a predetermined number of nuclei.

With reference, for example, to step (m), the protease inhibitor may include phenylmethanesulfonyl fluoride (PMSF), 4-(2-aminoethyl)benzenesulfonyl fluoride hydrochloride (AEBSF), a combination thereof, and/or another suitable protease inhibitor. With reference, for example, to steps (j), (k), (l), and/or (m), the capture agent may include biotin or another suitable capture agent. Furthermore, the binding agent may include avidin (e.g., streptavidin) or another suitable binding agent.

In certain embodiments, the methods of uniquely labeling RNA molecules within a plurality of nuclei may further include (e.g., after step (m)): (n) conducting a template switch of the cDNA molecules and AAV.RNAbc amplicons bound to the binding agent using a template switch oligonucleotide; (o) amplifying the cDNA molecules and AAV.RNAbc amplicons to form an amplified cDNA molecule and AAV.RNAbc amplicon solution; and/or (p) introducing a solid phase reversible immobilization (SPRI) bead solution to the amplified cDNA molecule and AAV.RNAbc amplicon solution to remove polynucleotides of less than about 200 base pairs, less than about 175 base pairs, or less than about 150 base pairs (see DeAngelis, M M, et al. Nucleic Acids Research (1995) 23(22):4742). In other words, the cDNA molecules and AAV.RNAbc amplicons can be bound to streptavidin beads within a lysate. Template switching of the cDNA molecules and AAV.RNAbc amplicons attached to the beads can be performed, e.g., to add an adapter to the 3-end of the cDNA molecules and AAV.RNAbc amplicons. PCR amplification of the cDNA molecules and AAV.RNAbc amplicons can then be performed, followed by the addition of SPRI beads to remove polynucleotides of less than about 200 base pairs. The ratio of SPRI bead solution to amplified cDNA molecule solution may be between about 0.9:1 and about 0.7:1, between about 0.875:1 and about 0.775:1, between about 0.85:1 and about 0.75:1, between about 0.825:1 and about 0.725:1, about 0.8:1, or another suitable ratio. Furthermore, the SPRI bead solution may include between about 1 M and 4 M NaCl, between about 2 M and 3 M NaCl, between about 2.25 M and 2.75 M NaCl, about 2.5 M NaCl, or another suitable amount of NaCl. The SPRI bead solution may also include between about 15% w/v and 25% w/v polyethylene glycol (PEG), wherein the molecular weight of the PEG is between about 7,000 g/mol and 9,000 g/mol (PEG 8000). In various embodiments, the SPRI bead solution may include between about 17% w/v and 23% w/v PEG 8000, between about 19% w/v and 22% w/v PEG 8000, between about 19% w/v and 21% w/v PEG 8000, about 20% w/v PEG 8000, or another suitable % w/v PEG 8000.

The methods of uniquely labeling RNA molecules within a plurality of nuclei may further include adding a common adapter sequence to the 3′-end of the released cDNA molecules and AAV.RNAbc amplicons. The common adapter sequence can be an adapter sequence that is the same, or substantially the same, for each of the cDNA molecules and AAV.RNAbc amplicons (i.e., within a given experiment). The addition of the common adapter may be conducted or performed in a solution including up to about 10% w/v of PEG, wherein the molecular weight of the PEG is between about 7,000 g/mol and 9,000 g/mol. In certain embodiments, the common adapter sequence may be added to the 3′-end of the released cDNA molecules and AAV.RNAbc amplicons by template switching (see Picelli, S. et al. Nature Methods 10, 1096-1098 (2013)).

The step (j) may be repeated a number of times sufficient to generate a unique series of barcode tags for the nucleic acids in a single nucleus. For example, the number of times can be selected from 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, and 100.

In various embodiments, the primers of step (b) may further include a first specific barcode. Stated another way, the first barcode added to the cDNA molecules and AAV.RNAbc amplicons in a specific container, mixture, reaction, receptacle, sample, well, or vessel may be predetermined (e.g., specific to the given container, mixture, reaction, receptacle, sample, well, or vessel). For example, 96 sets of different well-specific RT primers may be used (e.g., in a 96-well plate). Accordingly, if there are 96 samples or aliquots, each sample or aliquot can get a unique well-specific barcode.

In various embodiments, each of the barcode tags may include a first strand, wherein the first strand includes (i) a barcode sequence including a 3′ end and a 5′ end and (ii) a 3′ hybridization sequence and a 5′ hybridization sequence flanking the 3′ end and the 5′ end of the barcode sequence, respectively. Each of the barcode tags may also include a second strand, wherein the second strand includes (i) a first portion complementary to at least one of the 5′ hybridization sequence and the adapter sequence and (ii) a second portion complementary to the 3′ hybridization sequence.

The methods of uniquely labeling RNA molecules within a plurality of nuclei may further include ligating at least two (or more) of the barcode tags that are bound to the cDNA molecules and AAV.RNAbc amplicons. The ligation may be performed within the first plurality of nuclei.

The methods may further include removing unbound barcode tags. In some embodiments, the methods may include ligating at least two of the barcode tags that are bound to the released cDNA molecules and AAV.RNAbc amplicons. The majority of the nucleic acid tag-bound cDNA molecules and AAV.RNAbc amplicons from a single nucleus may include the same series of bound barcode tags.

The cDNA molecules may be formed or generated in an aliquot (e.g., a reaction mixture). The concentration of the first reverse transcription primer in the aliquot may be between about 0.5 μM and about 10 μM, between about 1 μM and about 7 μM, between about 1.5 μM and about 4 μM, between about 2 μM and about 3 μM, about 2.5 μM, or another suitable concentration. The concentration of the second reverse transcription primer in the aliquot may be between about 0.5 μM and about 10 μM, between about 1 μM and about 7 μM, between about 1.5 μM and about 4 μM, between about 2 μM and about 3 μM, about 2.5 μM, or another suitable concentration.

### III. SEQUENCING LIBRARY PREPARATION

In various embodiments, sequencing may be performed on various sequencing platforms that require preparation of a sequencing library. In the case of whole transcriptome sequencing, the preparation typically involves fragmenting the cDNA (sonication, nebulization or shearing), followed by cDNA repair and end polishing (blunt end or A overhang), and platform-specific adapter ligation. In one embodiment, the methods described herein can utilize next generation sequencing technologies (NGS), that allow multiple samples to be sequenced individually as genomic molecules (i.e., singleplex sequencing) or as pooled samples including indexed genomic molecules (e.g., multiplex sequencing) on a single sequencing run. These methods can generate up to several billion reads of DNA sequences. In various embodiments the sequences of genomic nucleic acids, and/or of indexed genomic nucleic acids can be determined using, for example, the Next Generation Sequencing Technologies (NGS) described herein. In various embodiments analysis of the massive amount of sequence data obtained using NGS can be performed using one or more processors.

Preparation of sequencing libraries for whole transcriptome sequencing is facilitated by the fragmentation of large polynucleotides (e.g. cDNA) to obtain polynucleotides in a desired size range.

Paired end reads may be used for the sequencing methods and systems disclosed herein. The fragment or insert length is longer than the read length, and sometimes longer than the sum of the lengths of the two reads.

In some illustrative embodiments, the sample nucleic acid(s) are obtained as cDNA, which is subjected to fragmentation into fragments of longer than approximately 50, 100, 200, 300, 400, 500, 600, 700, 800, 900, 1000, 2000, or 5000 base pairs, to which NGS methods can be readily applied. In some embodiments, the paired end reads are obtained from inserts of about 100-5000 bp. In some embodiments, the inserts are about 100-1000 bp long. These are sometimes implemented as regular short-insert paired end reads. In some embodiments, the inserts are about 1000-5000 bp long.

Fragmentation can be achieved by any of a number of methods known to those of skill in the art. For example, fragmentation can be achieved by mechanical means including, but not limited to nebulization, sonication and hydroshear, or by enzymatic means. However mechanical fragmentation typically cleaves the DNA backbone at C—O, P—O and C—C bonds resulting in a heterogeneous mix of blunt and 3′- and 5′-overhanging ends with broken C—O, P—O and/C—C bonds (see, e.g., Alnemri and Liwack, J Biol. Chem 265:17323-17333 [1990]; Richards and Boyer, J Mol Biol 11.327-240 [1965]) which may need to be repaired as they may lack the requisite 5′-phosphate for the subsequent enzymatic reactions, e.g., ligation of sequencing adapters, that are required for preparing DNA for sequencing.

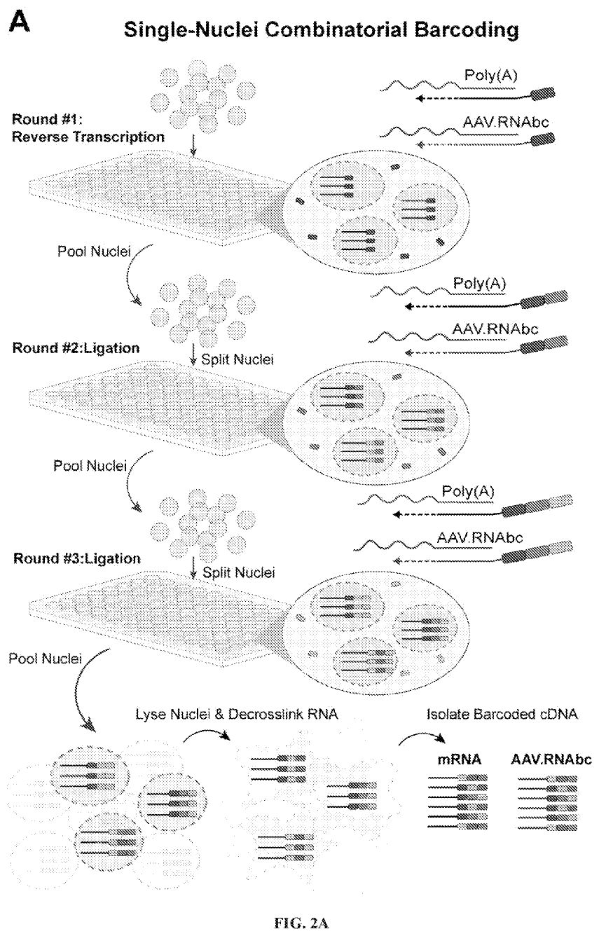

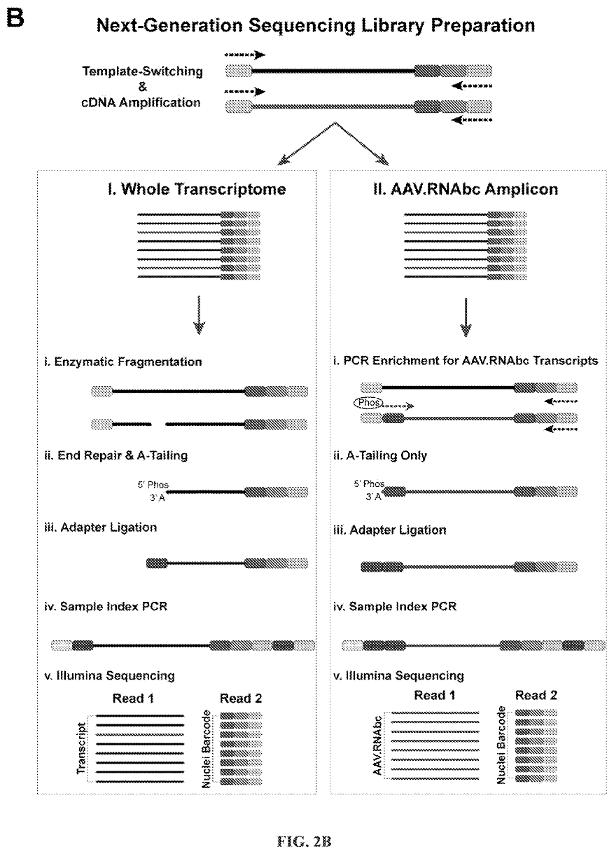

Typically, DNA fragments are converted to blunt-ended DNA having 5′-phosphates and 3′-hydroxyl. Standard protocols, e.g., protocols for sequencing using, for example, the Illumina platform as described in the example workflow in FIG. 2B, instruct users to end-repair sample DNA, to purify the end-repaired products prior to adenylating or dA-tailing the 3′ ends, and to purify the dA-tailing products prior to the adapter-ligating steps of the library preparation.

Various embodiments of methods of sequence library preparation described herein obviate the need to perform one or more of the steps typically mandated by standard protocols to obtain a modified DNA product that can be sequenced by NGS. For example, for enriched AAV.RNAbc amplicon sequencing, fragmentation is not performed. Rather, PCR enrichment is performed using a forward primer that is specific to the AAV.RNAbc amplicon, and that forward primer has a 5′ phosphate, thereby eliminating the need to perform end repair.

### IV. SEQUENCING METHODS

The methods and apparatus described herein may employ next generation sequencing technology (NGS), which allows massively parallel sequencing. In certain embodiments, clonally amplified DNA templates or single DNA molecules are sequenced in a massively parallel fashion within a flow cell (e.g., as described in Volkerding et al. Clin Chem 55:641-658 [2009]; Metzker M Nature Rev 11:31-46 [2010]). The sequencing technologies of NGS include but are not limited to pyrosequencing, sequencing-by-synthesis with reversible dye terminators, sequencing by oligonucleotide probe ligation, and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Examples of sequencing technologies that can be used to obtain the sequence information according to the present method are further described here.

Some sequencing technologies are available commercially, such as the sequencing-by-hybridization platform from Affymetrix Inc. (Sunnyvale, Calif.) and the sequencing-by-synthesis platforms from 454 Life Sciences (Bradford. Conn.), Illumina/Solexa (Hayward, Calif.) and Helicos Biosciences (Cambridge, Mass.), and the sequencing-by-ligation platform from Applied Biosystems (Foster City, Calif), as described below. In addition to the single molecule sequencing performed using sequencing-by-synthesis of Helicos Biosciences, other single molecule sequencing technologies include, but are not limited to, the SMRT™ technology of Pacific Biosciences, the ION TORRENT™ technology, and nanopore sequencing developed for example, by Oxford Nanopore Technologies.

While the automated Sanger method is considered as a ‘first generation’ technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM). Illustrative sequencing technologies are described in greater detail below.

In some embodiments, the disclosed methods involve obtaining sequence information for the nucleic acids in the test sample by massively parallel sequencing of millions of DNA fragments using Illumina's sequencing-by-synthesis and reversible terminator-based sequencing chemistry (e.g. as described in Bentley et al., Nature 6:53-59 [2009]). Template DNA can be cDNA. In some embodiments, cDNA from isolated cells is used as the template, and it is fragmented into lengths of several hundred base pairs. In other embodiments. AAV.RNAbc amplicons are prepared by PCR amplification, and fragmentation is not required. If needed, template DNA is end-repaired to generate 5′-phosphorylated blunt ends. The polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchor oligos. Under limiting-dilution conditions, adapter-modified, single-stranded template DNA is added to the flow cell and immobilized by hybridization to the anchor oligos. Attached DNA fragments are extended and bridge amplified to create an ultra-high density sequencing flow cell with hundreds of millions of clusters, each containing about 1,000 copies of the same template. In one embodiment, the adaptor-ligated DNA is amplified using PCR before it is subjected to cluster amplification. In some applications, the templates are sequenced using a robust four-color DNA sequencing-by-synthesis technology that employs reversible terminators with removable fluorescent dyes. High-sensitivity fluorescence detection is achieved using laser excitation and total internal reflection optics. Short sequence reads of about tens to a few hundred base pairs are aligned against a reference genome and unique mapping of the short sequence reads to the reference genome are identified using specially developed data analysis pipeline software. After completion of the first read, the templates can be regenerated in situ to enable a second read from the opposite end of the fragments. Thus, either single-end or paired end sequencing of the DNA fragments can be used.

Various embodiments of the disclosure may use sequencing by synthesis that allows paired end sequencing. In some embodiments, the sequencing by synthesis platform by Illumina involves clustering fragments. Clustering is a process in which each fragment molecule is isothermally amplified. In some embodiments, as the example described here, the fragment has two different adapters attached to the two ends of the fragment, the adapters allowing the fragment to hybridize with the two different oligos on the surface of a flow cell lane. The fragment further includes or is connected to two index sequences at two ends of the fragment, which index sequences provide labels to identify different samples in multiplex sequencing. In some sequencing platforms, a fragment to be sequenced from both ends is also referred to as an insert.

In some implementation, a flow cell for clustering in the Illumina platform is a glass slide with lanes. Each lane is a glass channel coated with a lawn of two types of oligos (e.g., P5 and P7′ oligos). Hybridization is enabled by the first of the two types of oligos on the surface. This oligo is complementary to a first adapter on one end of the fragment. A polymerase creates a compliment strand of the hybridized fragment. The double-stranded molecule is denatured, and the original template strand is washed away. The remaining strand, in parallel with many other remaining strands, is clonally amplified through bridge application.

In bridge amplification and other sequencing methods involving clustering, a strand folds over, and a second adapter region on a second end of the strand hybridizes with the second type of oligos on the flow cell surface. A polymerase generates a complementary strand, forming a double-stranded bridge molecule. This double-stranded molecule is denatured resulting in two single-stranded molecules tethered to the flow cell through two different oligos. The process is then repeated over and over, and occurs simultaneously for millions of clusters resulting in clonal amplification of all the fragments. After bridge amplification, the reverse strands are cleaved and washed off, leasing only the forward strands. The 3′ ends are blocked to prevent unwanted priming.

After clustering, sequencing starts with extending a first sequencing primer to generate the first read. With each cycle, fluorescently tagged nucleotides compete for addition to the growing chain. Only one is incorporated based on the sequence of the template. After the addition of each nucleotide, the cluster is excited by a light source, and a characteristic fluorescent signal is emitted. The number of cycles determines the length of the read. The emission wavelength and the signal intensity determine the base call. For a given cluster all identical strands are read simultaneously. Hundreds of millions of clusters are sequenced in a massively parallel manner. At the completion of the first read, the read product is washed away.

In the next step of protocols involving two index primers, an index 1 primer is introduced and hybridized to an index 1 region on the template. Index regions provide identification of fragments, which is useful for de-multiplexing samples in a multiplex sequencing process. The index 1 read is generated similar to the first read. After completion of the index 1 read, the read product is washed away and the 3′ end of the strand is de-protected. The template strand then folds over and binds to a second oligo on the flow cell. An index 2 sequence is read in the same manner as index 1. Then an index 2 read product is washed off at the completion of the step.

After reading two indices, read 2 initiates by using polymerases to extend the second flow cell oligos, forming a double-stranded bridge. This double-stranded DNA is denatured, and the 3′ end is blocked. The original forward strand is cleaved off and washed away, leaving the reverse strand. Read 2 begins with the introduction of a read 2 sequencing primer. As with read 1, the sequencing steps are repeated until the desired length is achieved. The read 2 product is washed away. This entire process generates millions of reads, representing all the fragments. Sequences from pooled sample libraries are separated based on the unique indices introduced during sample preparation. For each sample, reads of similar stretches of base calls are locally clustered. Forward and reversed reads are paired creating contiguous sequences.

### V. KITS

Another aspect of the disclosure relates to kits for labeling nucleic acids within at least a first cell. In some embodiments, the kit may comprise at least two reverse transcription primers comprising a 5′ overhang sequence. They kit may comprise at least one poly(T) comprising reverse transcription primer. The kit may comprise at least one AAV.RNAbc transcript-specific reverse transcription primer.

The kit may also comprise a plurality of first barcode tags. Each first barcode tag may comprise a first strand. The first strand may include a 3′ hybridization sequence extending from a 3′ end of a first labeling sequence and a 5′ hybridization sequence extending from a 5′ end of the first labeling sequence. Each first barcode tag may further comprise a second strand. The second strand may include an overhang sequence, wherein the overhang sequence may comprise (i) a first portion complementary to at least one of the 5′ hybridization sequence and the 5′ overhang sequence of the reverse transcription primer and (ii) a second portion complementary to the 3′ hybridization sequence.

The kit may further comprise a plurality of second barcode tags. Each second barcode tag may comprise a first strand. The first strand may include a 3′ hybridization sequence extending from a 3′ end of a second labeling sequence and a 5′ hybridization sequence extending from a 5′ end of the second labeling sequence. Each second barcode tag may further comprise a second strand. The second strand may comprise an overhang sequence, wherein the overhang sequence may comprise (i) a first portion complementary to at least one of the 5′ hybridization sequence and the 5′ overhang sequence of the reverse transcription primer and (ii) a second portion complementary to the 3′ hybridization sequence. In some embodiments, the first labeling sequence may be different from the second labeling sequence.

In some embodiments, the kit may also comprise one or more additional pluralities of barcode tags. Each barcode tag of the one or more additional pluralities of barcode tags may comprise a first strand. The first strand may include a 3′ hybridization sequence extending from a 3′ end of a labeling sequence and a 5′ hybridization sequence extending from a 5′ end of the labeling sequence. Each barcode tag of the one or more additional pluralities of barcode tags may also comprise a second strand. The second strand may include an overhang sequence, wherein the overhang sequence comprises (i) a first portion complementary to at least one of the 5′ hybridization sequence and the 5′ overhang sequence of the reverse transcription primer and (ii) a second portion complementary to the 3′ hybridization sequence. In some embodiments, the labeling sequence may be different in each given additional plurality of barcode tags.

In various embodiments, the kit may further comprise at least one of a reverse transcriptase, a fixation agent, a permeabilization agent, a ligation agent, and/or a lysis agent.

### VI. DEFINITIONS

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. A pol II promoter includes a minimal promoter that is a short DNA sequence comprised of a TATA-box and optionally other sequences that serve to specify the site of transcription initiation, to which regulatory elements are added for control of expression. A type I pol III promoter includes three cis-acting sequence elements downstream of the transcriptional start site: a) 5′sequence element (A block); b) an intermediate sequence element (I block); c) 3′ sequence element (C block). A type 2 pol III promoter includes two essential cis-acting sequence elements downstream of the transcription start site: a) an A box (5′ sequence element); and b) a B box (3′ sequence element). A type 3 pol III promoter includes several cis-acting promoter elements upstream of the transcription start site, such as a traditional TATA box, proximal sequence element (PSE), and a distal sequence element (DSE).

An “enhancer” is a DNA sequence that can stimulate transcription activity and may be an innate element of the promoter or a heterologous element that enhances the level or tissue specificity of expression. It is capable of operating in either orientation (5′->3′ or 3′->5′), and may be capable of functioning even when positioned either upstream or downstream of the promoter.

Promoters and/or enhancers may be derived in their entirety from a native gene, or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples of promoters include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, actin promoter, U6, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. In addition, sequences derived from intronic miRNA promoters, such as, for example, the miR107, miR206, miR208b, miR548f-2, miR569, miR590, miR566, and miR128 promoter, will also find use herein (see, e.g., Monteys et al., 2010). Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes a barcode, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

A nucleic acid/transgene is “operably linked” when it is placed into a functional relationship with another nucleic acid sequence. A nucleic acid/transgene encoding and barcode, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation. e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the invention, such polypeptides encoded by nucleic acid sequences are not required to be identical to the endogenous protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors, to the central nervous system, such as to distinct brain regions.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Design of a Double Barcode Containing AAV Cargo

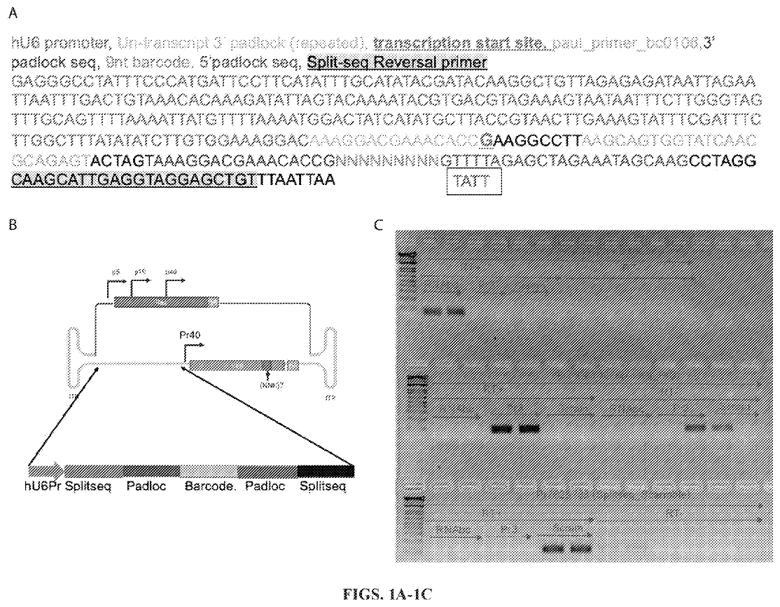

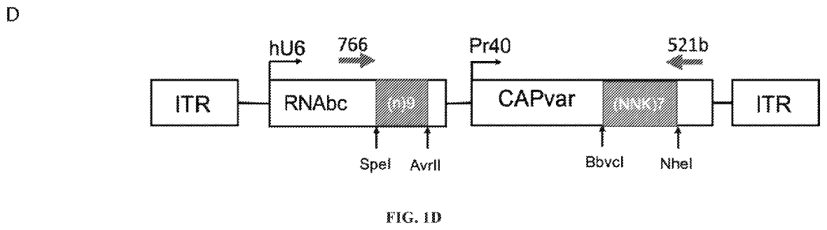

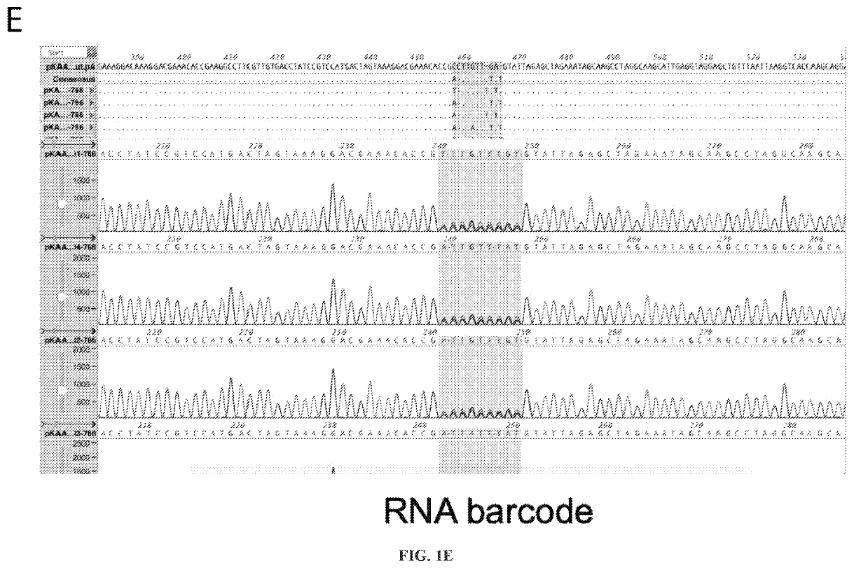

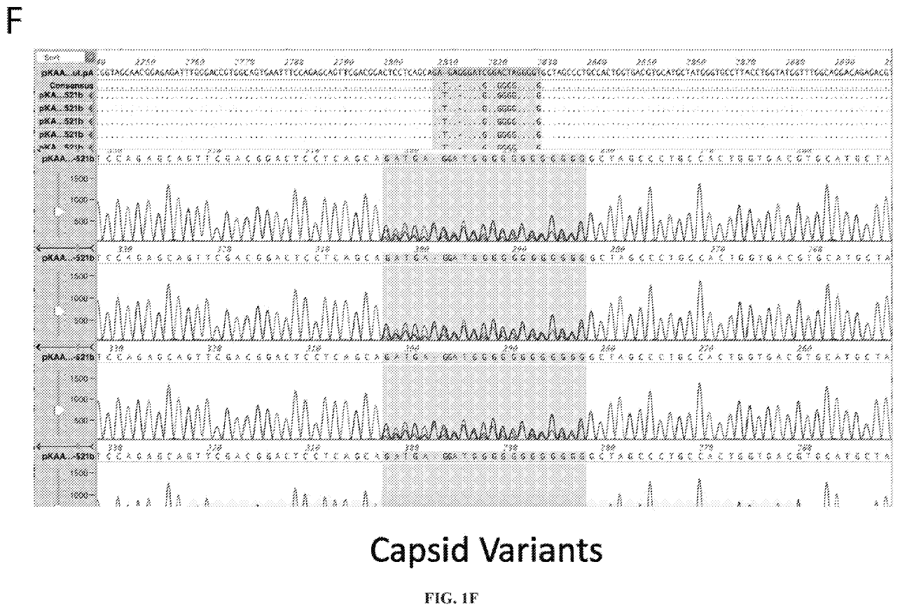

One of the primary challenges in detecting the transduction of barcoded AAV capsids at the single cell level is detecting both the mRNA sequences that provide information about cell identity and simultaneously detecting delivered AAV capsid DNA or expressed RNA. To overcome this challenge, the inventors engineered an AAV delivered expression construct that drives robust expression of a barcode sequence using the human U6 promoter (FIGS. 1A-B). The inventors designed the barcode sequence to be detectable by multiple methodologies including amplicon-sequencing, single-cell RNA-Sequencing. and in situ sequencing. The construct shown in FIG. 1A is packaged in AAV genome, as shown in FIGS. 1B&D. As an example, a full DNA sequence from such a construct is included in SEQ ID NO: 14, where nucleotides 1-141 are an AAV-1 ITR, nucleotides 148-404 are the human U6 promoter, nucleotides 187-207 are a binding site for Pr766, nucleotide 404 is the human U6 transcription start site, nucleotides 413-434 are a binding site for BC0108 scramble primer, nucleotides 441-456 are a 3′ padlock sequence, nucleotides 457-464 are a 9 nt RNA barcode, nucleotides 466-488 are a 5′ padlock sequence, nucleotides 495-516 are a binding site for Split-seq Pr Rev, nucleotides 525-705 are a Pr40 promoter, nucleotides 1028-3274 are the coding sequence of AAV1 capsid, nucleotides 2807-2827 are a coding sequence for (NNK)7 peptide, nucleotides 3117-3139 are a binding site for Pr521 Rev, and nucleotides 3408-3548 are an AAV2 ITR. In addition, the AAV Cap gene sequence has been modified to contain a peptide insertion. Critically, each modified Cap sequence is paired with a single RNA barcode “RNAbc”. These pairings are resolved by long-read sequencing capturing both the RNAbc and Cap insertion sequences. FIG. 1C provides an example of successful amplification of the RNAbc sequence exclusively after reverse transcription using primers pr749 and 750, in contrast to the non-specific amplification seen using other primer sets. Sanger sequencing spanning each insertion confirmed successful creation of this dual barcoded construct (FIGS. 1E&F).

TABLE 2
Primer sequences

| Primer | | SEQ |
|---|---|---|
| Name | Sequence (5′→3′) | ID NO: |
| Pr749 | CTAGTTCATGAGACCGGTG | 8 |
| Pr750 | TCAAGTTGATAACGGACTAGC | 9 |
| Pr761 | CGAACGTATTCTGATCGCATCGT | 10 |
| Pr738 | ACAGCTCCTACCTCAATGCTTG | 11 |
| Pr762 | CGTTGTGACCTATCCGTCCATG | 12 |
| Pr738 | ACAGCTCCTACCTCAATGCTTG | 13 |

| SEQ ID NO: 14 | |
|---|---|
| 1 | CCTGCAGGCA GCTGCGCGCT CGCTCGCTCA CTGAGGCCCC CCGGGCAAAG CCCGGGCGTC |
| 61 | GGGCGACCTT TGGTCGCCCG GCCTCAGTGA GCGAGCGAGC GCGCAGAGAG GGAGTGGCCA |
| 121 | ACTCCATCAC TAGGGGTTCC TACGCGTGAG GGCCTATTTC CCATGATTCC TTCATATTTG |
| 181 | CATATACGAT ACAAGGCTGT TAGAGAGATA ATTAGAATTA ATTTGACTGT AAACACAAAG |
| 241 | ATATTAGTAC AAAATACGTG ACGTAGAAAG TAATAATTTC TTGGGTAGTT TGCAGTTTTA |
| 301 | AAATTATGTT TTAAAATGGA CTATCATATG CTTACCGTAA CTTGAAAGTA TTTCGATTTC |
| 361 | TTGGCTTTAT ATATCTTGTG GAAAGGACAA AGGACGAAAC ACCGAAGGCC TTCGTTGTGA |
| 421 | CCTATCCGTC CATGACTAGT AAAGGACGAA ACACCGNNNV NNNVNGTATT AGAGCTAGAA |
| 481 | ATAGCAAGCC TAGGCAAGCA TTGAGGTAGG AGCTGTTTAA TTAAGGTCAC CAAGCAGGAA |
| 541 | GTCAAAGACT TTTTCCGGTG GGCAAAGGAT CACGTGGTTG AGGTGGAGCA TGAATTCTAC |
| 601 | GTCAAAAAGG GTGGAGCCAA GAAAAGACCC GCCCCCAGTG ACGCAGATAT AAGTGAGCCC |
| 661 | AAACGGGTGC GCGAGTCAGT TGCGCAGCCA TCGACGTCAG ACGCGGAAGC TTCGATCAAC |
| 721 | TACGCAGACA GGTACCAAAA CAAATGTTCT CGTCACGTGG GCATGAATCT GATGCTGTTT |
| 781 | CCCTGCAGAC AATGCGAGAG AATGAATCAG AATTCAAATA TCTGCTTCAC TCACGGACAG |
| 841 | AAAGACTGTT TAGAGTGCTT TCCCGTGTCA GAATCTCAAC CCGTTTCTGT CGTCAAAAAG |
| 901 | GCGTATCAGA AACTGTGCTA CATTCATCAT ATCATGGGAA AGGTGCCAGA CGCTTGCACT |
| 961 | GCCTGCGATC TGGTCAATGT GGATTTGGAT GACTGCATCT TTGAACAATA AATGATTTAA |
| 1021 | ATCAGGTATG GCTGCCGATG GTTATCTTCC AGATTGGCTC GAGGACAACC TCTCTGAGGG |
| 1081 | CATTCGCGAG TGGTGGGACT TGAAACCTGG AGCCCCGAAG CCCAAAGCCA ACCAGCAAAA |
| 1141 | GCAGGACGAC GGCCGGGGTC TGGTGCTTCC TGGCTACAAG TACCTCGGAC CCTTCAACGG |
| 1201 | ACTCGACAAG GGGGAGCCCG TCAACGCGGC GGACGCAGCG GCCCTCGAGC ACGACAAGGC |
| 1261 | CTACGACCAG CAGCTCAAAG CGGGTGACAA TCCGTACCTT CGGTATAACC ACGCCGACGC |
| 1321 | CGAGTTTCAG GAGCGTCTGC AAGAAGATAC GTCTTTTGGG GGCAACCTCG GGCGAGCAGT |
| 1381 | CTTCCAGGCC AAGAAGCGGG TTCTCGAACC TCTCGGTCTG GTTGAGGAAG GCGCTAAGAC |
| 1441 | GGCTCCTGGA AAGAAACGTC CGGTAGAGCA GTCGCCACAA GAGCCAGACT CCTCCTCGGG |
| 1501 | CATCGGCAAG ACAGGCCAGC AGCCCGCTAA AAAGAGACTC AATTTTGGTC AGACTGGCGA |
| 1561 | CTCAGAGTCA GTCCCCGACC CACAACCTCT CGGAGAACCT CCAGCAACCC CCGCTGCTGT |
| 1621 | GGGACCTACT ACAATGGCTT CAGGCGGTGG CGCACCAATG GCAGACAATA ACGAAGGCGC |
| 1681 | CGACGGAGTG GGTAATGCCT CAGGAAATTG GCATTGCGAT TCCACATGGC TGGGCGACAG |
| 1741 | AGTCATCACC ACCAGCACCC GCACCTGGGC CTTGCCCACC TACAATAACC ACCTCTACAA |
| 1801 | GCAAATCTCC AGTGCTTCAA CGGGGGCCAG CAACGACAAC CACTACTTCG GCTACAGCAC |
| 1861 | CCCCTGGGGG TATTTTGATT TCAACAGATT CCACTGCCAC TTTTCACCAC GTGACTGGCA |
| 1921 | GCGACTCATC AACAACAATT GGGGATTCCG GCCCAAGAGA CTCAACTTCA AACTCTTCAA |
| 1981 | CATCCAAGTC AAGGAGGTCA CGACGAATGA TGGCGTCACA ACCATCGCTA ATAACCTTAC |
| 2041 | CAGCACGGTT CAAGTCTTCT CGGACTCGGA GTACCAGCTT CCGTACGTCC TCGGCTCTGC |
| 2101 | GCACCAGGGC TGCCTCCCTC CGTTCCCGGC GGACGTGTTC ATGATTCCGC AATACGGCTA |
| 2161 | CCTGACGCTC AACAATGGCA GCCAAGCCGT GGGACGTTCA TCCTTTTACT GCCTGGAATA |
| 2221 | TTTCCCTTCT CAGATGCTGA GAACGGGCAA CAACTTTACC TTCAGCTACA CCTTTGAGGA |
| 2281 | AGTGCCTTTC CACAGCAGCT ACGCGCACAG CCAGAGCCTG GACCGGCTGA TGAATCCTCT |
| 2341 | CATCGACCAA TACCTGTATT ACCTGAACAG AACTCAAAAT CAGTCCGGAA GTGCCCAAAA |
| 2401 | CAAGGACTTG CTGTTTAGCC GTGGGTCTCC AGCTGGCATG TCTGTTCAGC CCAAAAACTG |
| 2461 | GCTACCTGGA CCCTGTTATC GGCAGCAGCG CGTTTCTAAA ACAAAAACAG ACAACAACAA |
| 2521 | CAGCAATTTT ACCTGGACTG GTGCTTCAAA ATATAACCTC AATGGGCGTG AATCCATCAT |
| 2581 | CAACCCTGGC ACTGCTATGG CCTCACACAA AGACGACGAA GACAAGTTCT TTCCCATGAG |
| 2641 | CGGTGTCATG ATTTTTGGAA AAGAGAGCGC CGGAGCTTCA AACACTGCAT TGGACAATGT |
| 2701 | CATGATTACA GACGAAGAGG AAATTAAAGC CACTAACCCG GTAGCAACGG AGAGATTTGG |
| 2761 | GACCGTGGCA GTGAATTTCC AGAGCAGTTC GACGGACTCC TCAGCANNKN NKNNKNNKNN |
| 2821 | KNNKNNKGCT AGCCCTGCCA CTGGTGACGT GCATGCTATG GGTGCCTTAC CTGGTATGGT |
| 2881 | TTGGCAGGAC AGAGACGTGT ACCTGCAGGG TCCCATTTGG GCCAAAATTC CTCACACAGA |
| 2941 | TGGACACTTT CACCCGTCTC CTCTTATGGG CGGCTTTGGA CTCAAGAACC CGCCTCCTCA |
| 3001 | GATCCTCATC AAAAACACGC CTGTTCCTGC GAATCCTCCG GCGGAGTTTT CAGCTACAAA |
| 3061 | GTTTGCTTCA TTCATCACCC AATACTCCAC AGGACAAGTG AGTGTGGAAA TTGAATGGGA |
| 3121 | GCTGCAGAAA GAAAACAGCA AGCGCTGGAA TCCCGAAGTG CAGTACACAT CCAATTATGC |
| 3181 | AAAATCTGCC AACGTTGATT TTACTGTGGA CAACAATGGA CTTTATACTG AGCCTCGCCC |
| 3241 | CATTGGCACC CGTTACCTTA CCCGTCCCCT GTAATTACGT GTTAATCAAT AAACCGGTTA |
| 3301 | ATTCGTGTCA GTTGAACTTT GGTCTCATGT CGTTATTATC TTATCTGGTC ACGAGATACG |
| 3361 | TAGATAAGTA GCATGGCTCT AGAGATCTGT GTGTTGGTTT TTTGTGTAGG AACCCCTAGT |
| 3421 | GATGGAGTTG GCCACTCCCT CTCTGCGCGC TCGCTCGCTC ACTGAGGCCG GGCGACCAAA |
| 3481 | GGTCGCCCGA CGCCCGGGCT TTGCCCGGGC GGCCTCAGTG AGCGAGCGAG CGCGCAGCTG |
| 3541 | CCTGCAGG |

### Example 2—Adaptation of Split-Pool Ligation-Based Whole-Transcriptome Sequencing (SPLiT-Seq) for AAV.RNAbc Detection

As shown in FIG. 2A, during three rounds of barcoding, fixed and permeabilized nuclei are distributed randomly into each well of a 96-well plate. Each well of the Round #1 barcoding plate corresponds to two barcoded primers: an oligo(dT) RT primer and an AAV-specific RT primer to capture AAV.RNAbc transcripts. During Round #1 both poly(A) and AAV.RNAbc transcripts from the same nucleus will be reverse transcribed and labeled with the same Round #1 barcode. All nuclei from the same well will receive the same Round #1 barcode, allowing for the encoding of sample information via Round #1 well position. After reverse transcription all nuclei are pooled and redistributed randomly in Round #2 and Round #3. The second and third rounds of barcoding consist of ligation reactions to add the additional single-nuclei barcodes. The third round of barcoding also adds a Unique Molecular Identified (UMI). After three rounds of barcoding in 96-well plates, 884,736 nuclei-barcode combinations are possible (96×96×6). All nuclei are pooled and split into sub-libraries of <10,000 nuclei prior to lysis, decrosslinking and streptavidin bead-based cDNA isolation.

FIG. 2B illustrates next-generation library preparation for whole-transcriptome and AAV.RNAbc amplicon sequencing. A template-switching reaction adds a 5′ common sequence for full-length cDNA amplification. Post-amplification, the libraries are split for whole-transcriptome and AAV.RNAbc amplicon sequencing from the same nuclei. Libraries for whole-transcriptome sequencing are fragmented, prior to undergoing end-repair. A-tailing, and adapter ligation (see FIG. 2B part I). A final PCR is performed to add Illumina adapters and dual indices. Paired-end Illumina sequencing is performed. Read 1 contains both mRNA and AAV.RNAbc sequence information and Read 2 corresponds to the single-nuclei barcode for downstream demultiplexing. To enrich for AAV.RNAbc sequences, a second PCR-based amplification is performed with 5′ primer sequence upstream of the AAV barcode (see FIG. 2B part II). In addition to enrichment, this step controls the size and start position of the AAV.RNAbc amplicon. The forward primer also is modified to add a phosphate to the PCR product, allowing for subsequent ligation of an Illumina sequencing adapter. A-tailing and adapter ligation are then performed prior to a final PCR to add Illumina adapters and sample indices. Paired-end Illumina sequencing is performed. Here Read 1 output corresponds to the AAV.RNAbc amplicon sequences and Read 2 to the single-nuclei barcode for downstream demultiplexing. Given that cDNA libraries from the same nuclei are split post-amplification and processed simultaneously through both library preparations, the single-nuclei barcodes are used to identify the cell-type identity of cells expressing AAV.RNAbc transcripts.

### Example 3—Detection of the RNA Barcode (RNAbc) Expressed after Transfection of HEK 293 Cells

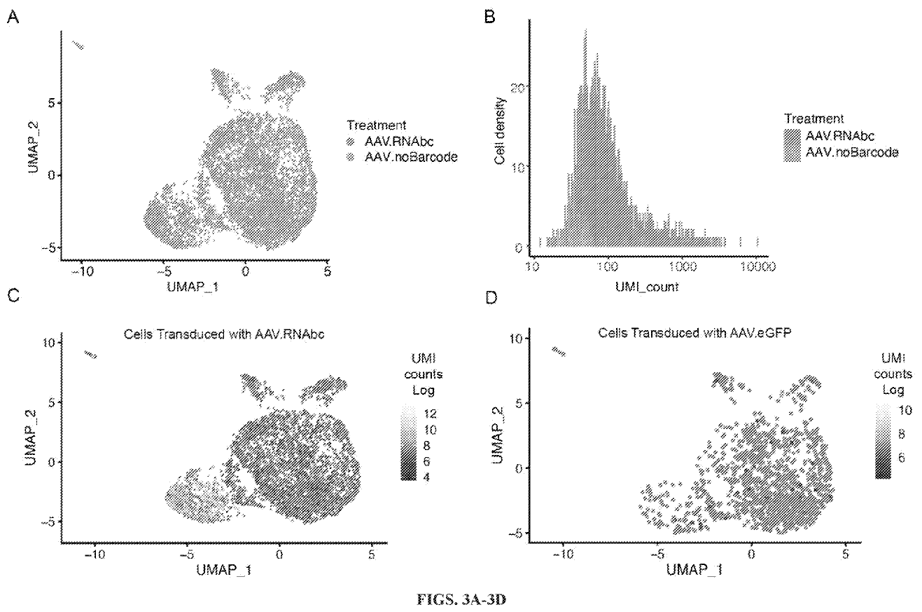

HEK 293 cells were transfected with plasmids containing either AAV.RNAbc or AAV.noBarcode. Uniform manifold approximation and projection (UMAP) unbiased clustering of the SPLiT-Seq barcoded single-cells is shown in FIG. 3A. Unique UMI counts were obtained from Illumina sequencing reads after amplification and library preparation exclusive to the RNAbc amplicon (FIG. 3B). As expected the vast majority of counts belong to single cells that received the AAV.RNAbc construct. A small minority of counts were found to originate from AAV.noBarcode treated cells, likely indicative of doublet nuclei. This is consistent with the 0.1% doublet rate of SPLiT-Seq. The Seurat single-cell object was subset to include only cells that received the AAV.RNAbc treatment (FIG. 3C) or only cells that received the AAV.eGFP construct (FIG. 3D). When the Seurat single-cell object was subset to include only cells that received the AAV.eGFP construct a background level of counts were observed to have originated from the AAV.RNAbc amplicon. Further filtering in non-monoculture contexts will help to remove this background.

### Example 4—In Vivo Detection of 67 AAV1 Capsid Variants in 1430 of 6739 Nuclei after AAV Variant Library Delivery to Mouse Brain

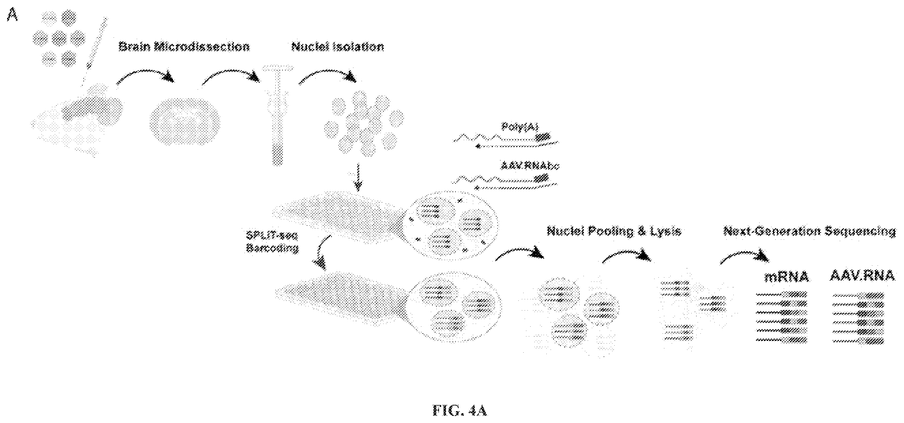

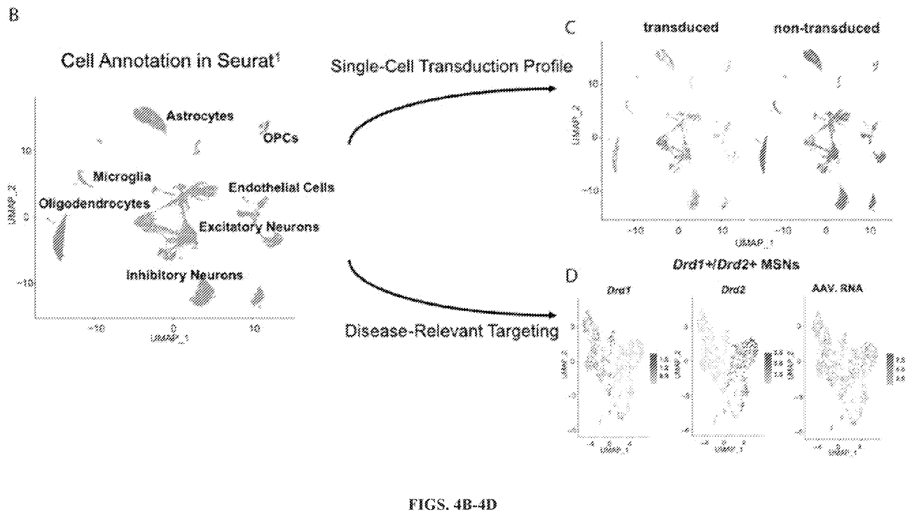

A library of 67 AAV1 capsid variants was delivered by intra-striatal and intra-thalamus injection into mouse brain. After incubation brain tissues were recovered and hippocampus, thalamus, striatum, and cortex regions were microdissected and carried forward into single nucleus isolation (FIG. 4A). Fixed and permeabilized nuclei were processed using the dual-Poly(A) and -AAV.RNAbc reverse transcription barcoding method disclosed herein. SPLiT-Seq based barcoding was then carried out to apply single-cell barcodes to the mRNAs and AAV transcripts contained inside the permeabilized nuclei. After the three single-cell barcodes were added nuclei were separated into two pools and lysed. In parallel, barcoded mRNAs and AAV.RNAs were then amplified and illumina indexes and sequencing adapters were added to facilitate sequencing on an illumina NovaSeq 6000.

The resulting fastq files were processed using a custom bioinformatic pipeline that integrates RNAbc sequence with AAV-peptide-insert information obtained from long-read sequencing of the input capsid library. This allowed the conversion of the detected RNA-bc to counts of AAV peptide inserts. Counts tables of cDNA counts per gene and RNA-bc counts per gene were produced and read into Seurat for downstream single-cell analysis and capsid transduction quantification.

Using the single cell barcodes that match between the cDNA and AAV derived datasets, the inventors were able to create a single Seurat object containing both cDNA expression and AAV transduction information (FIG. 4B). This allowed the inventors to determine which single cells have been transduced (FIG. 4C). By then looking at cell types relevant to disease, for example Drd1 and Drd2 positive medium spiny neurons (MSNs), the inventors were able to assess AAV transduction status within a single cell type of interest (FIG. 4D).

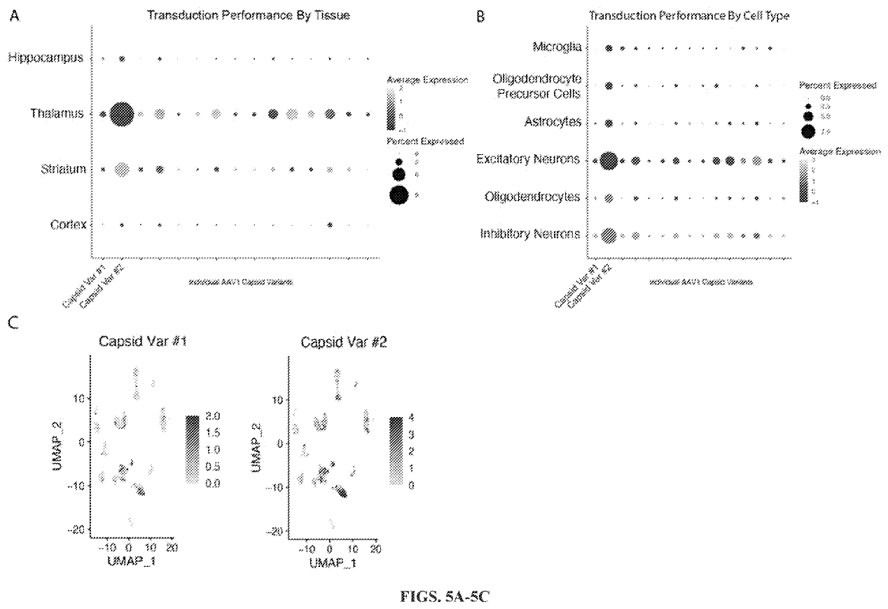

With this dataset and future datesets generated using this technology, the inventors are able to look with the tissues utilized for this study to identify which capsids perform best within a tissues of interest (FIG. 5A) and within individual cell types of interest (FIG. 5B). It is also possible to represent this information spatially using the UMAP unbiased clustering plots (FIG. 5C). These tools enable identification of capsid variants that have performance characteristics amenable to treatment of disease.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Rosenberg et al., “SPLiT-seq reveals cell types and lineages in the developing brain and spinal cord,” Science, 360:176-182, 2018.

What is claimed is:

1. A population of recombinant adeno-associated virus (rAAV) vectors, wherein each rAAV vector independently comprises (i) a modified adeno-associated virus (AAV) Cap gene encoding a modified AAV capsid protein comprising a targeting peptide and (ii) an expression cassette encoding a barcode sequence that is operably linked to an RNA polymerase III promoter, wherein the targeting peptide and the barcode in each rAAV vector are uniquely paired.

2. The population of rAAV vectors of claim 1, wherein the barcode sequence is 9-20 nucleotides long.

3. The population of rAAV vectors of claim 1 or 2, wherein the barcode sequence is (NNNT)ₙ.

4. The population of rAAV vectors of any one of claims 1-3, wherein the barcode sequence is flanked by sequences capable of hybridizing to and activating a padlock probe.

5. The population of rAAV vectors of any one of claims 1-4, wherein the RNA polymerase III promoter is a type III RNA polymerase III promoter.

6. The population of rAAV vectors of claim 8, wherein the RNA polymerase promoter is a U6 snRNA gene promoter, H1 RNA gene promoter, or 7SK gene promoter.

7. The population of rAAV vectors of any one of claims 1-6, further comprising a reverse transcription primer binding site positioned 3′ of the barcode sequence and an enrichment primer binding site positioned 5′ of the barcode sequence.

8. The population of rAAV vectors of any one of claims 1-7, wherein the expression cassette comprises a sequence that is at least 90% identical to SEQ ID NO: 7.

9. The population of rAAV vectors of any one of claims 1-8, wherein the modified AAV capsid protein is a modified AAV1 capsid protein, a modified AAV2 capsid protein, or a modified AAV9 capsid protein.

10. The population of rAAV vectors of claim 9, wherein the modified AAV capsid protein is derived from an AAV1 capsid protein (see SEQ ID NO: 1), wherein the targeting peptide is inserted after residue 590 of the AAV1 capsid protein.

11. The population of rAAV vectors of claim 10, wherein the targeting peptide is flanked by linker sequences, wherein the linker sequences on each side of the targeting peptides are two or three amino acids long.

12. The population of rAAV vectors of claim 11, wherein the linker sequences are SSA on the N-terminal side of the targeting peptide and AS on the C-terminal side of the targeting peptide.

13. The population of rAAV vectors of any one of claims 10-12, wherein the modified AAV1 capsid protein has a sequence at least 95% identical to SEQ ID NO: 4.

14. The population of rAAV vectors of claim 9, wherein the modified AAV capsid protein is derived from an AAV2 capsid protein (see SEQ ID NO: 2), wherein the targeting peptide is inserted after residue 587 of the AAV2 capsid protein.

15. The population of rAAV sectors of claim 14, wherein the targeting peptide is flanked by linker sequences, wherein the linker sequences on each side of the targeting peptides are two or three amino acids long.

16. The population of rAAV vectors of claim 15, wherein the linker sequences are AAA on the N-terminal side of the targeting peptide and AA on the C-terminal side of the targeting peptide.

17. The population of rAAV vectors of any one of claims 14-16, wherein the modified AAV2 capsid protein has a sequence at least 95% identical to SEQ ID NO: 5.

18. The population of rAAV vectors of claim 9, wherein the modified AAV capsid protein is derived from an AAV9 capsid protein (see SEQ ID NO: 3), wherein the targeting peptide is inserted after residue 588 of the AAV9 capsid protein.

19. The population of rAAV vectors of claim 18, wherein the targeting peptide is flanked by linker sequences, wherein the linker sequences on each side of the targeting peptides are two or three amino acids long.

20. The population of rAAV vectors of claim 19, wherein the linker sequences are AAA on the N-terminal side of the targeting peptide and AS on the C-terminal side of the targeting peptide.

21. The population of rAAV vectors of any one of claims 18-20, wherein the modified AAV9 capsid protein has a sequence at least 95% identical to SEQ ID NO: 6.

22. The population of rAAV vectors of any one of claims 8-21, wherein the targeting peptide is three to ten amino acids in length.

23. The population of rAAV vectors of claim 22, wherein the targeting peptide is seven amino acids in length.

24. The population of rAAV vectors according to any one of claims 8-23, wherein the population comprises a plurality of capsid protein targeting peptides, wherein each capsid protein targeting peptide is paired with more than one barcode sequence.

25. The population of rAAV vectors according to any one of claims 8-24, wherein the population comprises a plurality of capsid protein targeting peptides, wherein all rAAVs having the same barcode sequence also have the same capsid protein targeting peptide.

26. A population of cells comprising the population of rAAV vectors of any one of claims 1-25.

27. The population of cells of claim 26, wherein the cells are a mammalian cell.

28. The population of cells of claim 26, wherein the cells are a human cell.

29. The population of cells of claim 26, wherein the cells are in vitro.

30. The population of cells of claim 26, wherein the cells are in vivo.

31. A method of determining the cellular tropism of a recombinant adeno-associated virus (rAAV) having a modified AAV capsid protein comprising a targeting peptide, the method comprising (i) contacting a variety of cell types with the modified rAAV vectors of any one of claims 6-25; (ii) identifying cells transduced by the modified rAAV vectors based on the presence of the barcode sequence; and (iii) detecting the expressed transcriptome of each transduced cell, on a cell-by-cell basis, thereby determining the cellular tropism of the modified rAAV.

32. A method of determining the cellular tropism of a recombinant adeno-associated virus (rAAV) having a modified AAV capsid protein comprising a targeting peptide, the method comprising (i) contacting a variety of cell types with the population of rAAV vectors of any one of claims 6-25; (ii) detecting both the expressed transcriptome and the rAAV that transduced each cell, on a cell-by-cell basis; and (iii) determining which cell types were transduced by which modified rAAV vector, thereby determining the cellular tropism of the modified rAAV.

33. The method of claim 32, wherein the contacting in (i) is performed in vitro.

34. The method of claim 32, wherein the contacting in (i) is performed in vivo.

35. The method of any one of claims 32-34, wherein detecting the expressed transcriptome and the rAAV in (ii) comprises:
(a) isolating, fixing and permeabilizing the nuclei of the cells contacted in (i);
(b) dividing the nuclei into a plurality of first aliquots;
(c) reverse transcribing the expressed cellular RNA molecules within the nuclei using primers comprising a poly(T) sequence to form complementary DNA (cDNA) molecules, and reverse transcribing the expressed rAAV RNA molecules within the nuclei using primers comprising a sequence sufficient to hybridize to and reverse transcribe the barcode sequence within the expression cassette to form AAV amplicons;
(d) labeling the cDNA molecules and AAV amplicons with a first 5′ barcode, wherein the first 5′ barcode for the primers in each first aliquot is unique such that the cDNA molecules and AAV amplicons from the nuclei of each aliquot can be identified in comparison to the cDNA molecules and AAV amplicons from the nuclei of all other aliquots;
(e) combining the plurality of first aliquots;
(f) dividing the combined plurality of first aliquots into a plurality of second aliquots;
(g) ligating a second 5′ barcode to the 5′ ends of the cDNA molecules and the AAV amplicons for form dual barcoded cDNA molecules and AAV amplicons, wherein the second 5′ barcode in each second aliquot is unique;
(h) combining the plurality of second aliquots;
(i) dividing the combined plurality of first aliquots into a plurality of third aliquots;
(j) ligating a third 5′ barcode to the 5′ ends of the cDNA molecules and the AAV amplicons to form triple barcoded cDNA molecules and AAV amplicons, wherein the third 5′ barcode in each third aliquot is unique;
(k) combining the plurality of third aliquots;
(l) lysing the nuclei to release the cDNA molecules and the AAV amplicons from within the nuclei to form a lysate; and
(m) sequencing the cDNA molecules and the AAV amplicons to thereby detect both the expressed transcriptome and the rAAV that transduced each cell.

36. The method of claim 35, wherein the cDNA molecules and AAV amplicons are labeled with the first 5′ barcode simultaneously with the reverse transcription, wherein the reverse transcription primers comprising the first 5′ barcode.

37. The method of claim 35 or 36, wherein the nuclei are fixed and permeabilized at below about 8 °C, at below about 7 °C at below about 6 °C at below about 5 °C, at below about 4 °C, at below about 3 °C, at below about 2 °C, or at below about 1 °C.

38. The method of any one of claims 35-37, wherein the majority of the triple barcoded cDNA molecules and AAV molecules from a single nucleus comprise the same series of barcodes.

39. The method of claim 38, wherein the majority of the triple barcoded cDNA molecules and AAV molecules from a single nucleus have a unique series of barcodes as compared to the triple barcoded cDNA molecules and AAV molecules from other nuclei.

40. The method of any one of claims 32-39, wherein the cell types are determined based on the expressed transcriptome.

41. The method of any one of claims 32-40, wherein sequencing the cDNA molecules and the AAV amplicons comprises preparing a sequencing library, wherein preparing the sequencing library comprises:
(i) adding a common adapter sequence to the 3-ends of the cDNA molecules and AAV amplicons;
(ii) performing full-length cDNA and AAV amplicon amplification;
(iii) fragmenting the amplified full-length cDNA and AAV amplicons;
(iv) end repairing and A-tailing the fragmented cDNA and AAV amplicons;
(v) ligating an adaptor to the 5′ ends of the end repaired and A-tailed cDNA and AAV amplicons; and
(vi) performing a sample index PCR to add sequencing adapters and dual indices to the adaptor ligated cDNA and AAV amplicons.

42. The method of any one of claims 32-41, wherein sequencing the AAV amplicons comprises preparing a sequencing library enriched for the AAV amplicons, wherein the preparing the sequencing library comprises:
(i) adding a common adapter sequence to the 3-ends of the cDNA molecules and AAV amplicons;
(ii) performing full-length amplification of cDNA and AAV amplicons;
(iii) performing an AAV amplicon enrichment amplification with a forward primer that hybridizes to the AAV amplicons upstream of the AAV barcode, wherein the forward primer has a 5′ phosphate;
(iv) A-tailing the AAV amplicons having a 5′ phosphate;
(v) ligating an adaptor to the 5′ ends of the A-tailed AAV amplicons; and
(vi) performing a sample index PCR to add sequencing adapters and dual indices to the adaptor ligated AAV amplicons.

43. The method of claim 41 or 42, wherein the common adapter sequence is added to the 3′-end of the cDNA molecules and AAV amplicons by template switching.

44. The method of any one of claims 35-43, wherein the sequencing is paired-end sequencing, amplicon sequencing, single-cell RNA sequencing, or in situ sequencing.


SEQUENCE LISTING (21 sequences)

SEQ ID NO: 1 - protein, 736 aa, Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, Adeno-associated dependoparvovirus A [sequence omitted]

SEQ ID NO: 3 - protein, 736 aa, Adeno-associated dependoparvovirus A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDSSAXXXXXXXASPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - protein, 747 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAAXXXXXXXAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 6 - protein, 748 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAXXXXXXXASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - DNA, 377 bp, synthetic construct
  source 1..377: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaaaggacgaaacaccgaaggccttaagcagtggtatcaacgcagagtactagtaaaggacgaaacaccgnnnnnnnnngtttagagctagaaatagcaagcctaggcaagcattgaggtaggagctgtttaattaa

SEQ ID NO: 8 - DNA, 19 bp, synthetic construct
  source 1..19: mol_type = other DNA
ctagttcatgagaccggtg

SEQ ID NO: 9 - DNA, 21 bp, synthetic construct
  source 1..21: mol_type = other DNA
tcaagttgataacggactagc

SEQ ID NO: 10 - DNA, 23 bp, synthetic construct
  source 1..23: mol_type = other DNA
cgaacgtattctgatcgcatcgt

SEQ ID NO: 11 - DNA, 22 bp, synthetic construct
  source 1..22: mol_type = other DNA
acagctcctacctcaatgcttg

SEQ ID NO: 12 - DNA, 22 bp, synthetic construct
  source 1..22: mol_type = other DNA
cgttgtgacctatccgtccatg

SEQ ID NO: 13 - DNA, 22 bp, synthetic construct
  source 1..22: mol_type = other DNA
acagctcctacctcaatgcttg

SEQ ID NO: 14 - DNA, 3548 bp, synthetic construct
  source 1..3548: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaaaggacgaaacaccgaaggccttcgttgtgacctatccgtccatgactagtaaaggacgaaacaccgnnnvnnnvngtattagagctagaaatagcaagcctaggcaagcattgaggtaggagctgtttaattaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtaccttcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaacccggtagcaacggagagatttgggaccgtggcagtgaatttccagagcagttcgacggactcctcagcannknnknnknnknnknnknnkgctagccctgccactggtgacgtgcatgctatgggtgccttacctggtatggtttggcaggacagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattacgtgttaatcaataaaccggttaattcgtgtcagttgaactttggtctcatgtcgttattatcttatctggtcacgagatacgtagataagtagcatggctctagagatctgtgtgttggttttttgtgtaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 15 - DNA, 158 bp, synthetic construct
  source 1..158: mol_type = other DNA
gaaaggacaaaggacgaaacaccgaaggccttcgttgtgacctatccgtccatgactagtaaaggacgaaacaccgccttgttgagtattagagctagaaatagcaagcctaggcaagcattgaggtaggagctgtttaattaaggtcaccaagcagg

SEQ ID NO: 16 - DNA, 81 bp, synthetic construct
  source 1..81: mol_type = other DNA
acctatccgtccatgactagtaaaggacgaaacaccgtttgtttgtgtattagagctagaaatagcaagcctaggcaagca

SEQ ID NO: 17 - DNA, 81 bp, synthetic construct
  source 1..81: mol_type = other DNA
acctatccgtccatgactagtaaaggacgaaacaccgattgtttatgtattagagctagaaatagcaagcctaggcaagca

SEQ ID NO: 18 - DNA, 81 bp, synthetic construct
  source 1..81: mol_type = other DNA
acctatccgtccatgactagtaaaggacgaaacaccgattgtttgtgtattagagctagaaatagcaagcctaggcaagca

SEQ ID NO: 19 - DNA, 81 bp, synthetic construct
  source 1..81: mol_type = other DNA
acctatccgtccatgactagtaaaggacgaaacaccgattatttatgtattagagctagaaatagcaagcctaggcaagca

SEQ ID NO: 20 - DNA, 161 bp, synthetic construct
  source 1..161: mol_type = other DNA
cggtagcaacggagagatttgggaccgtggccagtgaatttccagagcagttcgacggactcctcagcagagagggatcggactaggggtgctagccctgccactggtgacgtgcatgctatgggtgccttacctggtatggtttggcaggacagagacgt

SEQ ID NO: 21 - DNA, 81 bp, synthetic construct
  source 1..81: mol_type = other DNA
tccagagcagttcgacggactcctcagcagatgaggatgggggggggggggctagccctgccactggtgacgtgcatgcta